(19) United States
(12) Patent Application Publication
(10) Pub. No.: US 2026/0152618 A1
(43) Pub. Date: Jun. 4, 2026

(54) PROCESSES FOR RECOVERING DIALKYL TEREPHTHALATES FROM POLYESTER COMPOSITIONS

(71) Applicant: Eastman Chemical Company, Kingsport, TN (US)

(72) Inventors: Pinguan Zheng, Johnson City, TN (US); Hannah Grace Harris, Kingsport, TN (US)

(73) Assignee: Eastman Chemical Company, Kingsport, TN (US)

(21) Appl. No.: 18/703,849

(22) PCT Filed: Oct. 25, 2022
(86) PCT No.: PCT/US2022/047656

§ 371 Date: Apr. 23, 2024

Related U.S. Application Data
(60) Provisional application No. 63/262,977, filed on Oct. 25, 2021.

(51) Int. Cl. C08G 18/10; C08G 63/16; C08G 63/183; C08G 63/78; C08L 67/02

(52) U.S. Cl. CPC C08J 11/24; C07C 67/03; C07C 67/48; C08J 2367/02

(57) ABSTRACT

Processes for recovering dialkyl terephthalates. The processes can include exposing a polyester composition to one or more glycols to depolymerization conditions thereby providing one or more depolymerization products. The one or more depolymerization products can be exposed to an alcoholysis process to recover dialkyl terephthalate. Optionally, the one or more glycols can be recycled and re-used in a subsequent dialkyl terephthalate recovery or other process.

### FIELD OF THE INVENTION

The present disclosure relates to processes for recycling polyester composition. More particularly, the present disclosure relates to recovering dialkyl terephthalates from polyester compositions.

### BACKGROUND OF THE INVENTION

Certain conventional systems may utilize glycolysis and/or methanolysis processes in an attempt to recycle polyesters. However, certain conventional glycolysis and/or methanolysis processes may require a substantial amount of resources and energy in order to arrive at suitable products for use in subsequent production processes, e.g., production processes to generate recycled polyesters or other compositions.

### BRIEF SUMMARY OF THE INVENTION

In one aspect, a process for recovering one or more dialkyl terephthalates from a polyester composition is provided. The process can include obtaining a polyester composition comprising one or more polyesters and exposing the polyester composition to one or more glycols under depolymerization conditions to provide a first mixture. The first mixture can comprise a first liquid component and a first solid component, the first liquid component comprising one or more depolymerization products. A weight ratio of the amount of the one or more glycols relative to the amount of the polyester composition can be from 4:1 to 1:9. The process can also include separating at least a portion of the first liquid component from the first solid component. The separating can occur at a temperature of from 50° C. to 150° C. The process can further include exposing the at least a portion of the first liquid component to an alcohol composition under conditions including a temperature of from 23° C. to 90° C. to provide a second mixture. The second mixture can comprise a second liquid component and a second solid component, the second solid component comprising one or more dialkyl terephthalates.

In another aspect, a process for recovering one or more dialkyl terephthalates from a polyester composition is provided. The process can include obtaining a polyester composition comprising one or more polyesters and exposing the polyester composition to one or more glycols under depolymerization conditions to provide a first mixture. The first mixture can comprise a first liquid component and a first solid component, the first liquid component comprising one or more depolymerization products, wherein the one or more depolymerization products comprise monomers, oligomers, or a combination thereof. The process can also include separating at least a portion of the first liquid component from the first solid component, where the separating occurs at a temperature of 150° C. or less. Further, the process can include exposing the at least a portion of the first liquid component to an alcohol composition under conditions including a temperature of 90° C. or less to provide a second mixture. The second mixture can comprise a second liquid component and a second solid component, the second solid component comprising one or more dialkyl terephthalates.

In yet another aspect, a process for recovering one or more dialkyl terephthalates from a polyester composition is provided. The process can include exposing a first polyester composition to one or more glycols under depolymerization conditions to provide a first mixture. The first mixture can comprise a first liquid component and a first solid component, the first liquid component comprising one or more depolymerization products. The process can also include exposing at least a portion of the first liquid component to a first alcohol composition under alcoholysis conditions comprising a temperature of 90° C. or less to provide a second mixture. The second mixture can comprise a second liquid component and a second solid component, the second solid component comprising one or more dialkyl terephthalates. The process can further include exposing the second liquid component to distillation conditions to provide one or more recycle glycols, the one or more recycle glycols comprising at least a portion of the one or more glycols. Further, the process can include exposing a second polyester composition to at least a portion of the one or more recycle glycols under depolymerization conditions to provide a third mixture. The third mixture can comprise a third liquid component and a third solid component, the third liquid component comprising one or more depolymerization products.

### BRIEF DESCRIPTION OF THE DRAWING

FIG. 1 is an example system for recovering one or more dialkyl terephthalates from a polyester composition, in accordance with aspects of the present disclosure.

FIG. 2 is another example system for recovering one or more dialkyl terephthalates from a polyester composition, in accordance with aspects of the present disclosure.

FIG. 3 is a Gel Permeation Chromatography (GPC) curve at 255 nanometers for various samples from Examples 2A and 2D showing the oligomeric distribution of the samples, in accordance with aspects of the present disclosure.

FIG. 4 is a graph depicting the impurity distribution of various filtrate/wash samples from Example 12.

FIG. 5 is a graph depicting the impurity distribution of various filtrate/wash samples from Examples 7A-7D.

### DETAILED DESCRIPTION OF THE INVENTION

### Overview

The present disclosure may be understood more readily by reference to the following detailed description of certain aspects of the disclosure and working examples. In according with the purpose(s) of this disclosure, certain aspects of the disclosure are described in the Brief Summary of the Invention and are further described herein below. Also, other aspects of the disclosure are described herein.

Aspects herein are directed to processes for recovering one or more dialkyl terephthalates from polyester compositions. As described herein, an example process can include exposing a polyester composition to one or more glycols under depolymerization conditions to generate one or more depolymerization products, which are then exposed to an alcoholysis process for recovery of dialkyl terephthalate.

As discussed above, certain conventional glycolysis and/or methanolysis processes may require a substantial amount of resources and energy in order to arrive at suitable products for use in subsequent production processes, e.g., production processes to generate recycled polyesters or other compositions.

The processes and systems disclosed herein can alleviate one or more of the above problems. For instance, in certain aspects, the processes disclosed herein can include exposing a polyester composition to depolymerization conditions with one or more glycols to provide one or more depolymerization products. In various aspects, the one or more depolymerization products can include monomers, oligomers, or a combination thereof. In aspects, the one or more depolymerization products can be exposed to alcoholysis conditions resulting in a dialkyl terephthalate product of high yield and purity. As discussed herein, the alcoholysis conditions include a temperature that is reduced compared to certain conventional systems, which reduces the overall energy and resources required. In aspects, as discussed herein, the depolymerization and alcoholysis conditions described herein are substantially milder than certain conventional processes, which results in less ethylene glycol yield loss, e.g., due to fewer side reactions or degradation reactions converting ethylene glycol into various impurities. Further, in certain aspects as discussed further below, glycols present in the resulting alcoholysis liquid component can be separated from at least a portion of the alcohol composition utilized in the alcoholysis, and these recycle glycols can be re-used in subsequent rounds of dialkyl terephthalate recovery, which also reduces resource consumption.

### Polyester Compositions

As discussed above, the processes described herein relate to recovering one or more dialkyl terephthalates from a polyester composition. The term “polyester” can refer to a synthetic polymer prepared by the reaction of one or more difunctional carboxylic acids and/or multifunctional carboxylic acids with one or more difunctional hydroxyl compounds and/or multifunctional hydroxyl compounds. The difunctional carboxylic acid can be a dicarboxylic acid and the difunctional hydroxyl compound can be a dihydric alcohol such as, for example, glycols. Furthermore, as used herein, the term “diacid” or “dicarboxylic acid” includes multifunctional acids, such as branching agents. The term “glycol” or “diol” as used herein, includes, but is not limited to, diols, glycols, and/or multifunctional hydroxyl compounds. The dicarboxylic acid residues may be derived from a dicarboxylic acid monomer or its associated acid halides, esters, salts, anhydrides, or mixtures thereof. As used herein, therefore, the term dicarboxylic acid is intended to include dicarboxylic acids and any derivative of a dicarboxylic acid, including its associated acid halides, esters, halfesters, salts, half-salts, anhydrides, mixed anhydrides, or mixtures thereof, useful in a reaction process with a diol to make polyester. It should be understood, that the term “polyester” as used herein also refers to copolyesters.

As used herein, the term “residue(s)” refers to the monomer unit or repeating unit in a polymer, oligomer, or dimer. For example, a polymer can be made from the condensation of the following monomers: terephthalic acid (“TPA”) and cyclohexyl-1,4-dimethanol (“CHDM”). The condensation reaction results in the loss of water molecules. The residues in the resulting polymer are derived from either terephthalic acid or cyclohexyl-1,4-dimethanol. Below in Formula (I), a non-limiting example of a polyester is provided.

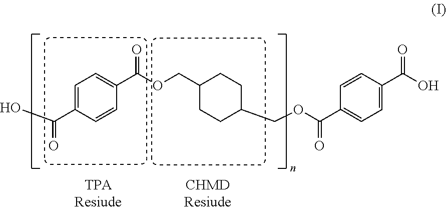

In aspects, the polyester composition exhibits an inherent viscosity of from about 0.1 dL/g to about 1.2 dL/g as determined according to ASTM D2857-70, about 0.2 dL/g to about 1.2 dL/g as determined according to ASTM D2857-70, about 0.3 dL/g to about 1.2 dL/g as determined according to ASTM D2857-70, or about 0.4 dL/g to about 1.2 dL/g as determined according to ASTM D2857-70.

In aspects, the polyester composition can include one or more polyesters. In various aspects, the one or more polyesters can include terephthalate polyesters. Terephthalate polyesters are polyesters that comprise residues of terephthalic acid or residues of any derivative of terephthalic acid, including its associated acid halides, esters, half-esters, salts, half-salts, anhydrides, mixed anhydrides, and/or mixtures thereof or residues thereof useful in a reaction process with a diol to make a copolyester In various aspects, the polyester composition can include polyethylene terephthalate (PET). In one or more aspects, the polyester composition can include glycol-modified PET. In certain aspects, the polyester composition can include polyethylene terephthalate (PET), 1,4-cyclohexanedimethanol (CHDM)-modified PET, isophthalic acid (IPA)-modified PET, diethylene glycol (DEG)-modified PET, glycol-modified PET, neopentyl glycol (NPG)-modified PET, propane diol (PDO)-modified PET, butanediol (BDO)-modified PET, heaxanediol (HDO)-modified PET, 2-methyl-2,4-pentanediol (MP diol)-modified PET, isosorbide-modified PET, poly(tetramethylene ether) glycol (PTMG)-modified PET, poly(ethylene) glycol (PEG)-modified PET, polycyclohexylenedimethylene terephthalate (PCT), cyclohexanedimethanol (CHDM)-containing copolyester, isosorbide-containing copolyester, or a combination thereof. In the same or alternative aspect, the polyester composition can include polyethylene terephthalate (PET) that comprises CHDM, IPA, DEG, NPG, PDO, BDO, HDO, MP diol, isosorbide, PTMG, PEG, or a combination thereof.

In various aspects, the polyester composition can include CHDM. In one aspect, the polyester composition can include about 0 mole % to about 100 mole % CHDM, about 1 mole % to about 100 mole % CHDM, about 1 mole % to about 90 mole % CHDM, about 1 mole % to about 80 mole % CHDM, about 1 mole % to about 70% CHDM, about 1 mole % to about 60 mole % CHDM, about 1 mole % to about 50 mole % CHDM, about 1 mole % to about 40 mole % CHDM, about 1 mole % to about 35 mole % CHDM, about 1 mole % to about 30 mole % CHDM, about 1 mole % to about 25 mole % CHDM, about 1 mole % to about 20 mole % CHDM, about 1 mole % to about 10 mole % CHDM, or about 1 mole % to about 5 mole % CHDM. In aspects, the mole % of CHDM refers to the mole % of CHDM relative to all diol equivalents in the polyester composition. In various aspects, the polyester composition can include DEG. In aspects, the polyester composition can include about 0 mole % to about 100 mole % DEG, about 1 mole % to about 100 mole % DEG, about 1 mole % to about 90 mole % DEG, about 1 mole % to about 80 mole % DEG, about 1 mole % to about 70 mole % DEG, about 1 mole % to about 60 mole % DEG, about 1 mole % to about 50 mole % DEG, about 1 mole % to about 40 mole % DEG, about 1 mole % to about 35 mole % DEG, about 1 mole % to about 30 mole % DEG, about 1 mole % to about 20 mole % DEG, about 1 mole % to about 10 mole % DEG, about 1 mole % to about 5 mole % DEG, or about 1 mole % to about 3 mole % DEG. In aspects, the mole % of DEG refers to the mole % of DEG relative to all diol equivalents in the polyester composition. In aspects, the polyester composition can include isophthalic acid. In aspects, the polyester composition can include about 0 mole % to about 30 mole % isophthalic acid, about 1 mole % to about 30 mole % isophthalic acid, about 1 mole % to about 25 mole % isophthalic acid, about 1 mole % to about 20 mole % isophthalic acid, about 1 mole % to about 15 mole % isophthalic acid, about 1 mole % to about 10 mole % isophthalic acid, about 1 mole % to about 7.5 mole % isophthalic acid, about 1 mole % to about 5 mole % isophthalic acid, about 1 mole % to about 3 mole % isophthalic acid, about 10 mole % or less of isophthalic acid, about 7.5 mole % or less of isophthalic acid, about 5 mole % or less of isophthalic acid, or about 3 mole % or less of isophthalic acid. In aspects, the mole % of isophthalic acid refers to the mole % of isophthalic acid relative to all diacid equivalents in the polyester composition In certain aspects, the polyester composition can include about 0 mole % to about 100 mole % CHDM, about 0 mole % to about 100 mole % DEG, about 0 mole % to about 30 mole % isophthalic acid, or a combination thereof. In certain aspects, the polyester composition can include about 1 mole % to about 100 mole % CHDM, about 1 mole % to about 100 mole % DEG, about 1 mole % to about 30 mole % isophthalic acid, or a combination thereof. In various aspects, the polyester composition can include other glycols, e.g., other than those mentioned above. For instance, in aspects, the polyester composition can include, but is not limited to, neopentyl glycol (NPG), 2-methyl-2,4-pentanediol (MP diol), butanediol (BDO), propanediol (PDO), hexanediol (HDO), isosorbide, poly(tetramethylene ether) glycol (PTMG), poly(ethylene) glycol (PEG), or a combination thereof. In certain aspects, each of the NPG, MP diol, BDO, PDO, HDO, isosorbide, PTMG, and PEG can be present in the polyester composition in an amount of 0 mole % to about 100 mole %, about 1 mole % to about 100 mole %, about 1 mole % to about 90 mole %, about 1 mole % to about 80 mole %, about 1 mole % to about 70%, about 1 mole % to about 60 mole %, about 1 mole % to about 50 mole %, about 1 mole % to about 40 mole %, about 1 mole % to about 35 mole %, about 1 mole % to about 30 mole %, about 1 mole % to about 25 mole %, about 1 mole % to about 20 mole %, about 1 mole % to about 10 mole %, or about 1 mole % to about 5 mole %. In aspects, the mole % of each of NPG, MP diol, BDO, PDO, HDO, isosorbide, PTMG, and PEG refers to the mole % of each of NPG, MP diol, BDO, PDO, HDO, isosorbide, PTMG, and PEG, respectively, relative to all diol equivalents in the polyester composition. In various aspects, the polyester composition can include CHDM, DEG, NPG, MP diol, BDO, PDO, HDO, isosorbide, PTMG, PEG, isophthalic acid, or a combination thereof, where each component is present in any of the amounts for such components described in this paragraph.

In aspects, the polyester composition or the one or more polyesters present in the polyester composition can be recycled polyesters. In various aspects, the recycled polyester(s) can include material that was recovered as manufacturing scrap, industrial waste, post-consumer waste, or a combination thereof. In aspects, the recycled polyester(s) can be prior-used products that have been used and/or discarded. In aspects, the polyester composition and/or recycled polyester(s) can come from various sources and/or in various forms, including but not limited to textiles, carpet, thermoformed materials, bottles, pellets, and film. In one or more aspects, the polyester composition can include renewal polyesters, e.g., polyesters formed from DMT recovered from a prior DMT recovery process, such as the processes described herein.

In various aspects, the polyester composition can include one or more foreign materials. In aspects, the one or more foreign materials may include, but are not limited to, polyesters other than polyethylene terephthalate, polyvinyl chloride (PVC), polyvinyl acetal, polyvinyl butyral (PVB), polyvinyl alcohol (PVOH), ethylene vinyl alcohol (EVOH), cotton, polyolefins, polyethylene, polypropylene, polystyrene, polycarbonate, Spandex, natural fibers, cellulose ester, polyacrylates, polymethacrylate, polyamides, nylon, poly(lactic acid), polydimethylsiloxane, polysilane, calcium carbonate, titanium dioxide, inorganic fillers, dyes, pigments, color toners, colorants, plasticizers, adhesives, flame retardants, metals, aluminum, and iron, or a combination thereof. In various aspects, the one or more foreign materials can be present in the polyester composition in an amount of from about 0.01 wt. % to about 50 wt. %, about 0.01 wt. % to about 40 wt. %, about 0.01 wt. % to about 30 wt. %, about 0.01 wt. % to about 20 wt. %, about 0.01 wt. % to about 15 wt. %, about 0.01 wt. % to about 10 wt. %, about 0.01 wt. % to about 7.5 wt. %, about 0.01 wt. % to about 5 wt. %, about 0.01 wt. % to about 2.5 wt. %, about 0.01 wt. % to about 1.0 wt. %, relative to the weight of the one or more polyesters in the polyester composition.

In aspects, the polyester composition can be in solid form, liquid form, molten form, or in a solution. In certain aspects, the solution can include a polyester composition pre-dissolved in a solvent, e.g., DMT, EG, DEG, TEG, or a combination thereof.

### Optional Pre-Treatment of the Polyester Composition

In certain aspects, an optional treatment of the polyester composition, prior to glycolysis and/or methanolysis, can be performed. In various aspects, the optional pretreatment can include any type of treatment that aids in removing a portion of any foreign materials from the polyester composition and/or that aids in recovering one or more polyesters from a mixed feedstock, e.g., a feedstock comprising the foreign materials discussed above. For instance, in one aspect, the optional pretreatment can include exposing to the polyester composition to one or more solvents, in an effort to selectively dissolve the polyester in the polyester composition (or at least a portion of the foreign materials in the polyester composition) to allow for separation between at least a portion of the foreign materials and the one or more polyesters in the polyester composition. As one example aspect, the optional pretreatment can include exposing the polyester composition to one or more solvents, e.g., one or more solvents that can cause dissolution of the polyester in the polyester composition. For instance, the one or more solvents can include but are not limited to 4-methylcyclohexanemethanol (MCHM), ethylene glycol (EG), diethylene glycol (DEG), triethylene glycol (TEG), 1,4-cyclohexanedimethanol (CHDM), poly(ethylene glycol) (PEG), neopentyl glycol (NPG), propane diol (PDO), butanediol (BDO), 2-methyl-2,4-pentanediol (MP diol), poly(tetramethylene ether)glycol (PTMG), dibutyl terephthalate (DBT), dioctyl terephthalate (DOTP), ethylene carbonate (EC), dimethyl carbonate (DMC), dimethyl sulfoxide (DMSO), dimethylformamide (DMF), or combinations thereof. In the same or alternative aspects, the polyester composition can be exposed to the one or more solvents at specific temperatures to effectuate dissolution of one or more components. In various aspects, a pretreatment process can include one or more dissolution and separation steps using various solvents and/or temperatures to achieve a desired level of foreign materials removal and/or purity level of PET. For instance, in one aspect, a dissolution and separation can be utilized using one solvent at a specific temperature, e.g., to remove one or more foreign materials, followed by a subsequent dissolution and separation of the polyester fraction using another solvent at a specific temperature, e.g., to remove one or more other foreign materials. The dissolution and/or separation(s) in this optional pretreatment step can utilize any suitable systems, reactors, vessels, and/or separation techniques to achieve a desired pretreated polyester composition.

### Glycolysis of the Polyester Composition

As discussed above, in various aspects, the processes disclosed herein can include exposing a polyester composition to depolymerization conditions to depolymerize at least a portion of the one or more polyesters into one or more depolymerization products. In various aspects, the one or more depolymerization products can include monomers, oligomers, or a combination thereof. In certain aspects, the oligomers can exhibit a degree of polymerization from 2 to 10, 2 to 8, 2 to 6, or 2 to 4. In aspects, the one or more polyesters may be depolymerized into one or more depolymerization products that can include monomers and terephthalate oligomers having a degree of polymerization from 2 to 10, 2 to 8, 2 to 6, or 2 to 4. In aspects, liquid chromatography can be utilized to discern the degree of polymerization of an oligomer, and/or gel permeation chromatography can be utilized to discern the molecular weight of the oligomers.

In aspects, the term degree of polymerization (DP) can refer to the number of residues in the oligomer. As used herein, the degree of polymerization (DP) refers to the number of difunctional carboxylic acid residues and/or multifunctional carboxylic acid residues in the oligomer. For instance, in one example aspect, a DP of one, would refer to a residue that includes one terephthalic acid residue or one isophthalic acid residue. In such an example aspect, a DP of one can also be referred to as a monomer. A non-limiting example of a DP of one is provided below in formula (II).

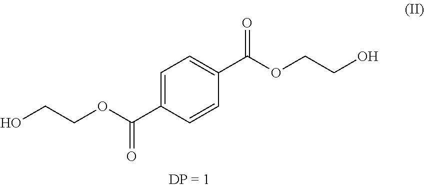 [SMILES: O=C(OCCO)c1ccc(C(=O)OCCO)cc1]

Formulas (III)-(V) below show non-limiting examples of oligomers having a DP of two, three, and n, respectively, in aspects.

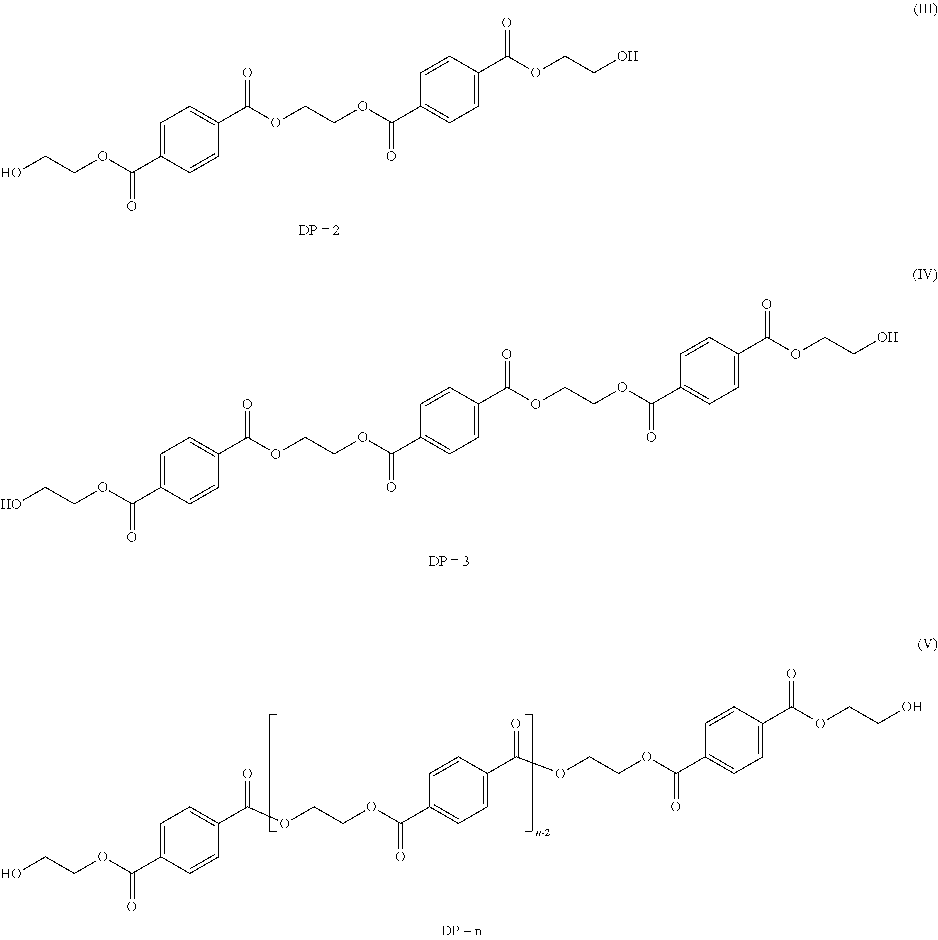

In aspects, this depolymerization can occur via a glycolysis process. Generally, in aspects, the glycolysis process can include exposing a polyester composition to one or more glycols, where the glycols react with the polyester, optionally in the presence of a trans-esterification catalyst, forming a mixture of bis(hydroxyethyl) terephthalate (BHET) and low molecular weight terephthalate oligomers. Some representative examples of glycolysis methods are disclosed in U.S. Pat. Nos. 3,257,335; 3,907,868; 6,706,843; and 7,462,649, and are incorporated by reference herein.

In one aspect of a glycolysis process, one or more polyesters, e.g., one or more recycled polyesters, and one or more glycols can be fed into a glycolysis reactor where the one or more recycled polyesters are dissolved and depolymerized under depolymerization conditions.

In aspects, any amount of the one or more glycols suitable for use in a glycolysis process can be utilized. In various aspects, the weight ratio of the one or more glycols relative to the amount of the polyester composition can be of from 12:1 to 1:12, 8:1 to 1:9, 6:1 to 1:9, 4:1 to 1:9, 4:1 to 1:7, 4:1 to 1:4, 4:1 to 1:2, 3:1 to 1:9, 3:1 to 1:7, 3:1 to 1:4, 3:1 to 1:2, 2:1 to 1:9, 2:1 to 1:7, 2:1 to 1:4, 2:1 to 1:2, 4:1 to 2:7, 3:1 to 1:4, 3:1 to 1:3, 2:1 to 1:2, 2:1 to 3:7, 1:1 to 3:7, 4:1 to 3:7, 4:1 to 4:7, 4:1 to 5:7, or 3:1 to 3:7.

In certain aspects, the one or more glycols can include any glycol suitable for use in a glycolysis process. As used herein, the term “glycol” refers to aliphatic, alicyclic, and aralkyl glycols. Exemplary glycols include ethylene glycol, 1,2-propandiol (also known propylene glycol), 1,3-propanediol, 1,4-butanediol, 1,5-pentanediol, 1,6-hexanediol, 2,2-dimethyl-1,3-propanediol, 1,2-cyclohexane dimethanol, 1,3-cyclohexane dimethanol, 1,4-cyclohexane dimethanol, 2,2,4,4-tetramethyl-1,3-cyclobutanediol, isosorbide, p-xylylenediol, and the like. These glycols may also contain ether linkages, such as is the case in, for example, diethylene glycol, triethylene glycol, and tetraethylene glycol. Additional embodiments of glycols include higher molecular weight homologs, known as polyethylene glycols, such as those produced by Dow Chemical Company under the Carbowax™ tradename. In one embodiment, the polyethylene glycol has a molecular weight of from greater than 200 to about 10,000 Daltons (Mₙ). These glycols also include higher order alkyl analogs, such as dipropylene glycol, dibutylene glycol, and the like. Similarly, further glycols include higher order polyalkylene ether diols, such as polypropylene glycol and polytetramethylene glycol with molecular weights ranging from about 200 to about 10,000 Daltons (Mₙ) (also referred to as g/mole). In one aspect, the glycol can be chosen from aliphatic, alicyclic, and aralkyl glycols. In the same or alternative aspects, the glycol can be chosen from ethylene glycol; 1,2-propandiol; 1,3-propanediol; 1,4-butanediol; 1,5-pentanediol; 1,6-hexanediol; 2,2-dimethyl-1,3-propanediol; 1,2-cyclohexane dimethanol; 1,3-cyclohexane dimethanol; 1,4-cyclohexane dimethanol; 2,2,4,4-tetramethyl-1,3-cyclobutanediol; isosorbide; p-xylylenediol; diethylene glycol; triethylene glycol; tetraethylene glycol; polyethylene glycols; dipropylene glycol; dibutylene glycol; polyalkylene ether diols chosen from polypropylene glycol and polytetramethylene glycol.

In various aspects, the one or more glycols can include ethylene glycol (EG), diethylene glycol (DEG), triethylene glycol (TEG), 1,4-cyclohexanedimethanol (CHDM), poly(ethylene glycol) (PEG), neopentyl glycol (NPG), propane diol (PDO), butanediol (BDO), 2-methyl-2,4-pentanediol (MP diol), poly(tetramethylene ether)glycol (PTMG), or a combination thereof. In one aspect, the one or more glycols can include about 0 wt. % to about 100 wt. % EG, or about 1 wt. % to about 100 wt. % EG, relative to the total weight of the one or more glycols. In certain aspects, the one or more glycols can include about 0 wt. % to about 100 wt. % DEG, or about 1 wt. % to about 100 wt. % DEG, relative to the total weight of the one or more glycols. In certain aspects, the one or more glycols can include about 0 wt. % to about 100 wt. % TEG, or about 1 wt. % to about 100 wt. % TEG, relative to the total weight of the one or more glycols. In certain aspects, the one or more glycols can include about 0 wt. % to about 100 wt. % PEG, or about 1 wt. % to about 100 wt. % PEG, relative to the total weight of the one or more glycols. In certain aspects, the one or more glycols can include about 0 wt. % to about 100 wt. % NPG, or about 1 wt. % to about 100 wt. % NPG, relative to the total weight of the one or more glycols. In certain aspects, the one or more glycols can include about 0 wt. % to about 100 wt. % PDO, or about 1 wt. % to about 100 wt. % PDO, relative to the total weight of the one or more glycols. In certain aspects, the one or more glycols can include about 0 wt. % to about 100 wt. % BDO, or about 1 wt. % to about 100 wt. % BDO, relative to the total weight of the one or more glycols. In certain aspects, the one or more glycols can include about 0 wt. % to about 100 wt. % MP diol, or about 1 wt. % to about 100 wt. % MP diol, relative to the total weight of the one or more glycols. In certain aspects, the one or more glycols can include about 0 wt. % to about 100 wt. % PTMG, or about 1 wt. % to about 100 wt. % PTMG, relative to the total weight of the one or more glycols. In aspects, the one or more glycols can include about 0 wt. % to about 50 wt. % CHDM, or about 1 wt. % to about 50 wt. % CHDM, relative to the total weight of the one or more glycols. In one aspect, the one or more glycols can include 0 wt. % to about 100 wt. % EG, 0 wt. % to about 100 wt. % DEG, 0 wt. % to about 100 wt. % TEG, 0 wt. % to about 100 wt. % PEG, 0 wt. % to about 100 wt. % NPG, 0 wt. % to about 100 wt. % PDO, 0 wt. % to about 100 wt. % BDO, 0 wt. % to about 100 wt. % MP diol, 0 wt. % to about 100 wt. % PTMG, and 0 wt. % to about 50 wt. % CHDM, relative to the total weight of the one or more glycols. In one aspect, the one or more glycols can include 1 wt. % to about 100 wt. % EG, 1 wt. % to about 100 wt. % DEG, 1 wt. % to about 100 wt. % TEG, 1 wt. % to about 100 wt. % PEG, 1 wt. % to about 100 wt. % NPG, 1 wt. % to about 100 wt. % PDO, 1 wt. % to about 100 wt. % BDO, 1 wt. % to about 100 wt. % MP diol, 1 wt. % to about 100 wt. % PTMG, and 1 wt. % to about 50 wt. % CHDM, relative to the total weight of the one or more glycols. In certain aspects, as discussed in detail below, the one or more glycols can be recycle glycols that were recovered from a prior glycolysis and methanolysis process for recovery of one or more dialkyl terephthalates, as disclosed herein.

In various aspects, as discussed above, the glycolysis process can include one or more catalysts, e.g., trans-esterification catalysts. In certain aspects, the catalyst can be present in an amount of from 0.1 wt. % to 10 wt. %, relative to the weight of the polyester composition. In aspects, any suitable catalyst can be utilized. In one aspect, the catalyst can include a carbonate catalyst, for example, but not limited to: Li₂CO₃, K₂CO₃, Na₂CO₃, Cs₂CO₃, ZrCO₃, or a combination thereof. In one aspect, the catalyst can include a hydroxide catalyst, for example, but not limited to: LiOH, NaOH, KOH, tetrabutylammonium hydroxide (TBAH), or a combination thereof. In one aspect, the catalyst can include an alkoxide catalyst, for example, but not limited to: sodium methoxide (NaOMe), lithium methoxide (LiOMe), magnesium methoxide, potassium t-butoxide, ethylene glycol monosodium salt, ethylene glycol disodium salt, or a combination thereof. In one aspect, the catalyst can include tetra isopropyl titanate (TIPT), butyltin tris-2-ethylhexanoate (FASCAT 4102), 1,8-Diazabicyclo[5.4.0]undec-7-ene (DBU), zinc acetylacetonate hydrate (Zn(acac)₂), zinc acetate (Zn(OAc)₂), and manganese (II) acetate (Mn(OAc)₂)), or a combination thereof. In certain aspects, the catalyst can include LiOH, NaOH, KOH, tetra isopropyl titanate (TIPT), butyltin tris-2-ethylhexanoate (FASCAT 4102), ZrCO₃, 1,8-Diazabicyclo[5.4.0]undec-7-ene (DBU), sodium methoxide (NaOMe), lithium methoxide (LiOMe), and zinc acetylacetonate hydrate (Zn(acac)₂), or a combination thereof. In one aspect, the catalyst can include LiOH, NaOH, KOH, sodium methoxide (NaOMe), and lithium methoxide (LiOMe). In certain aspects, the catalyst can include Li₂CO₃, K₂CO₃, CaCO₃, Na₂CO₃, Cs₂CO₃, ZrCO3, LiOH, NaOH, KOH, tetrabutylammonium hydroxide (TBAH), sodium methoxide (NaOMe), lithium methoxide (LiOMe), magnesium methoxide (Mg(OMe)₂, potassium t-butoxide, ethylene glycol monosodium salt, ethylene glycol disodium salt, tetra isopropyl titanate (TIPT), butyltin tris-2-ethylhexanoate (FASCAT 4102), 1,8-Diazabicyclo[5.4.0]undec-7-ene (DBU), zinc acetylacetonate hydrate (Zn(acac)₂), zinc acetate (Zn(OAc)₂), manganese (II) acetate (Mn(OAc)₂), hydrotalcite, zeolite, lithium chloride, or a combination thereof. The depolymerization conditions can include a temperature of from 150° C. to 260° C. and an absolute pressure of from 1 atmosphere (atm) to 15 atm, or 1 atm to 2 atm, in an agitated reactor for 0.5 h to 10 h. Higher temperatures may be used to increase the rate of depolymerization; however, reactor systems that can withstand elevated pressures may be required. One or a plurality of reactors may be used for the reaction of the polyester with the one or more glycols. For example, the reaction mixture can be continuously withdrawn from the first stage and introduced to a second stage maintained under pressure, along with additional glycol, wherein depolymerization continues to the desired degree of completion. In various aspects, any type of vessel, reactor, and/or reactor system can be utilized for the depolymerization or glycolysis of the polyester composition. In one aspect, a continuous stirred-tank reactor or vessel, a fixed bed reactor, or a melt extruder. In the same or alternative aspects, the depolymerization or glycolysis of the polyester composition can be a batch or continuous process.

Upon exposure to the depolymerization conditions detailed above, the resulting mixture can optionally be allowed to cool to a temperature of about 150° C. or less, or of from about 50° C. to about 150° C. In aspects, the resulting mixture can be allowed to cool to the desired temperature in the glycolysis reaction vessel(s) or can be transferred to a different vessel for temperature reduction. The resulting mixture can include a solid component and a liquid component. In aspects, the liquid component includes the one or more depolymerization products, e.g., monomers and/or oligomers having a degree of polymerization of from 2 to 10, along with the one or more glycols, and may also include any additional soluble components from the polyester composition, one or more glycols, catalysts, or a combination thereof. In various aspects, the solid component can be the residual foreign materials and any other insoluble components of the polyester composition and may be considered a waste product to discard.

As discussed further below, the liquid component is further subjected to at least a methanolysis process for the recovery of one or more dialkyl terephthalates. In various aspects, prior to the methanolysis process, the liquid component can be separated from the solid component. In aspects, the liquid component can be separated from the solid component using any system. In one aspect, the liquid component can be separated from the solid component while the resulting mixture is at a temperature of from about 50° C. to about 150° C. In such aspects, separating the liquid component from the solid component at temperatures of about 150° C. or less, e.g., at about 50° C. to about 150° C., can provide for a more efficient process and/or less resource intensive process than current conventional processes. In the same or alternative aspects, separating the liquid component from the solid component at temperatures of about 150° C. or less, e.g., at about 50° C. to about 150° C., can be beneficial as some impurities can be unstable at higher temperatures, e.g., temperatures above 150° C., which may adversely affect the processes, product yields, and/or product purities described herein.

In various aspects, the separation of the liquid component from the solid component can include a filtration process. In such an aspect, any suitable filtration process can be utilized that is capable of withstanding the increased filtration temperatures of from about 50° C. to about 150° C. In certain aspects, the solid component can be removed by centrifugation. In certain aspects, the solid can be removed by settling or sedimentation. In certain aspects, the solid component may have settled in the bottom of a vessel allowing for removal of the liquid component through a vessel conduit or valve appropriately positioned within the vessel. In one aspects, such a conduit and/or valve may include a filtration device to minimize the inclusion of solid component in downstream processes.

### Alcoholysis of the One or More Depolymerization Products

As discussed above, in aspects, the one or more depolymerization products produced in the glycolysis process described above can be subjected to an alcoholysis process. Generally, in a typical alcoholysis process, a polyester is reacted with an alcohol, e.g., methanol, to produce a depolymerized mixture comprising oligomers, terephthalate monomers, e.g., dimethyl terephthalate (DMT), and one or more glycols. In other embodiments, other monomers such as, for example, CHDM, DEG, and dimethyl isophthalate (DMI), also may be produced, depending on the composition of the polyester. In one embodiment, during the alcoholysis process the terephthalate oligomers are reacted with methanol to produce a depolymerized polyester mixture comprising polyester oligomers, DMT, CHDM, and/or EG.

Some representative examples of the methanolysis of PET are described in U.S. Pat. Nos. 3,321,510; 3,776,945; 5,051,528; 5,298,530; 5,414,022; 5,432,203; 5,576,456; 6,262,294; which are incorporated herein by reference.

In aspects, the alcoholysis process can include exposing the liquid component and/or the one or more depolymerization products resulting from the glycolysis process to an alcohol composition under conditions resulting in one or more dialkyl terephthalates. As discussed above, in aspects, the one or more depolymerization products can be present in the liquid component resulting from the glycolysis process. In various aspects, as discussed above, prior to subjecting the one or more depolymerization products and/or the liquid component resulting from the glycolysis process to an alcoholysis process, the liquid component can be separated from the resulting mixture and/or from the resulting solid component of the glycolysis process. In certain aspects, after separating the liquid component from the solid component of the glycolysis process, the liquid component can be directly utilized in this alcoholysis process. In the same or alternative aspects, after separating the liquid component from the solid component of the glycolysis process, the liquid component is not subjected to any further processing, e.g., distillation and/or other separation processes, prior to being utilized in this alcoholysis process. Without being bound by any particular theory, it is believed that since the glycolysis process is performed using a lower amount of glycols compared to certain conventional processes (or a weight ratio of glycols relative to the amount of the polyester composition of from 3:1 to 1:9) allows for the resulting liquid component from the glycolysis process to be directly utilized in the alcoholysis process without requiring further processing, e.g., to concentrate the resulting one or more depolymerization products and/or remove a portion of the glycols.

The alcohol composition can include any suitable alcohol known in the art for use in an alcoholysis process to obtain a specific dialkyl terephthalate. In one aspect, the alcohol composition can be methanol. In aspects, when methanol is utilized as the alcohol composition, DMT can be the resulting methanolysis product.

In certain aspects, the amount of the alcohol composition can be any amount that is in excess on a weight basis relative to the amount or weight of the polyester composition. In certain aspects, a weight ratio of the amount of the alcohol composition relative to the amount of the polyester composition can be from about 2:1 to about 10:1. In such aspects, the amount of the polyester composition refers to the amount or weight of the polyester composition that is utilized in the above glycolysis process.

In aspects, the alcoholysis reaction can occur at a temperature of about 90° C. or less, about 80° C. or less, about 70° C. or less, about 60° C. or less, about 50° C. or less, about 40° C. or less, or about 30° C. or less. In the same or alternative aspects, the alcoholysis reaction can occur at a temperature of from about 20° C. to about 90° C., about 20° C. to about 80° C., about 20° C. to about 70° C., about 20° C. to about 60° C., about 20° C. to about 50° C., about 20° C. to about 40° C., or about 20° C. to about 30° C. In various aspects, without being bound by any particular theory, it is believed that, since in the processes disclosed herein, the polyester in the polyester composition has already undergone at least a partial depolymerization process, e.g., in the glycolysis step discussed above, that the methanolysis process can be performed at the temperatures described above, which are comparably reduced compared to certain other conventional processes. Additionally or alternatively, without being bound by any particular theory, it is believed that, since the one or more depolymerization products produced in the glycolysis process are separated from the waste or insoluble material prior to this alcoholysis process, the alcoholysis process can be conducted at the reduced temperatures described above.

In aspects, the alcoholysis process can be conducted in any suitable reactor and/or vessel. In aspects, the alcoholysis reactor can be in fluid communication with the reactor utilized in the glycolysis process described above. In certain aspects, the alcoholysis reactor is a different reactor than the vessel used for glycolysis. Alternatively, in various aspects, the alcoholysis process can be conducted in the same vessel as the glycolysis process and/or the filtration process discussed above. In certain aspects, the alcoholysis process can be conducted at ambient pressure, e.g., about 1 atm, or at a pressure of from about 1 atm to about 5 atm, or of from about 1 atm to about 3 atm. In various aspects, the alcoholysis reaction can be conducted at a pressure above ambient pressure, e.g., more than 1 atm, or about 5 atm or less, about 3 atm or less, when the alcoholysis reaction temperature is high for the process conditions disclosed herein, e.g., about 50° C. or more, about 60° C. or more, about 70° C. or more, about 80° C. or more, or about 90° C. or more.

In various aspects, an alcoholysis catalyst can be utilized in the alcoholysis process. In aspects, the alcoholysis catalyst can be present in an amount of from about 0.1 wt. % to about 20 wt. % relative to the weight of the polyester composition, or of from about 0.1 wt. % to about 10 wt. % relative to the weight of the polyester composition, or of from about 0.1 wt. % to about 5 wt. % relative to the weight of the polyester composition, or of from about 0.1 wt. % to about 2 wt. % relative to the weight of the polyester composition, or of from about 0.1 wt. % to about 1 wt. % relative to the weight of the polyester composition, or of from about 0.1 wt. % to about 0.5 wt. % relative to the weight of the polyester composition. In such aspects, the amount of the polyester composition refers to the amount or weight of the polyester composition that is utilized in the above glycolysis process. In various aspects, the alcoholysis catalyst amounts disclosed in this paragraph refer to the amount of alcoholysis catalyst present during the alcoholysis reaction. In various aspects, the alcoholysis catalyst amounts disclosed in this paragraph refer to the amount of alcoholysis catalysts that is added to one or more depolymerization products and the one or more alcohols to facilitate the alcoholysis reaction. In certain aspects, reduced or lower amounts of alcoholysis catalyst may be added to the one or more depolymerization products and the one or more alcohols to facilitate the alcoholysis reaction, such as an amount of from about 0.1 wt. % to about 10 wt. % relative to the weight of the polyester composition, or of from about 0.1 wt. % to about 5 wt. % relative to the weight of the polyester composition, or of from about 0.1 wt. % to about 2 wt. % relative to the weight of the polyester composition, or of from about 0.1 wt. % to about 1 wt. % relative to the weight of the polyester composition, or of from about 0.1 wt. % to about 0.5 wt. % relative to the weight of the polyester composition. In aspects, such a lower amount of alcoholysis catalysts may be added at least partly because alcoholysis catalyst is already present in the one or more depolymerization products and/or one or more alcohols. In such aspects, as discussed below, the alcohol and/or glycol may be recycled and re-used in subsequent glycolysis and alcoholysis process as disclosed herein, which may include at least a portion of alcoholysis catalyst from a prior alcoholysis and/or glycolysis process.

In various aspects, the alcoholysis catalyst can include a carbonate catalyst, for example, but not limited to: K₂CO₃, Na₂CO₃, Li₂CO₃, Cs₂CO₃; a hydroxide catalyst, for example, but not limited to: KOH, LiOH, NaOH; an alkoxide catalyst, for example, but not limited to NaOMe, Mg(OMe)₂, KOMe, KOt-Bu, ethylene glycol monosodium salt, ethylene glycol disodium salt, or a combination thereof. In certain aspects, the alcoholysis catalyst can include KOH, NaOH, LiOH, or a combination thereof. In certain aspects, the alcoholysis catalyst can include NaOMe, KOMe, Mg(OMe)₂, KOt-Bu, ethylene glycol monosodium salt, ethylene glycol disodium salt, or a combination thereof. In various aspects, the alcoholysis catalyst can be in solid form, a solution form in water, methanol, or ethylene glycol, or a combination of thereof. In certain aspects, the alcoholysis catalyst can be added to the one or more depolymerization products and the alcohol composition once the alcohol composition and the one or more depolymerization products reach the desired reaction temperature or temperature range disclosed above.

The one or more depolymerization products can be exposed to the alcohol composition and optionally the alcoholysis catalyst under the temperature and pressure conditions described above for a period of time to achieve the desired yield of the resulting dialkyl terephthalate. In certain aspects, the one or more depolymerization products can be exposed to the alcohol composition and optionally the alcoholysis catalyst under the temperature and pressure conditions described above for a period of time of from about 5 minutes to about 5 hours, or of from about 5 minutes to about 2 hours, or about 5 minutes to about 1 hour, or about 5 minutes to about 30 minutes, or about 5 minutes to about 15 minutes, or about 5 minutes to about 10 minutes.

In aspects, the alcoholysis process results in a mixture that includes one or more dialkyl terephthalates. In various aspects, the alcoholysis process results in mixture wherein the dialkyl terephthalate is an insoluble and/or solid component. In aspects, the liquid component of this mixture can include glycols, the alcohol composition, DEG, CHDM, or a combination thereof. In one aspect, the glycols can be the glycols that were utilized in the glycolysis process and present with the one or more depolymerization products at the initiation of the alcoholysis process. In various aspects, the dialkyl terephthalate can be isolated from the mixture using any known separation technique, e.g., filtering, centrifugation, sedimentation, settling, or a combination of one or more separation techniques. In aspects, the filtering may include washing the solid component with additional alcohol composition or other solvent. The resulting liquid component can include the filtrate and wash. The resulting solid component can include about 90 wt. % or more dialkyl terephthalate, e.g., DMT, about 93 wt. % or more dialkyl terephthalate, e.g., DMT, or about 95 wt. % or more dialkyl terephthalate, e.g., DMT, relative to the weight of the solid component. In the same or alternative aspects, the dialkyl terephthalate, e.g., DMT, in the resulting solid component can be about 90% or more pure, about 93% or more pure, or about 95% or more pure. In various aspects, the solid component can also include dimethyl isophthalate (DMI). In such aspects, the DMI can be present in an amount of about 1000 ppm or less, or about 500 ppm or less, or of from about 1 ppm to about 1000 ppm, or of from about 1 ppm to about 500 ppm. In one or more aspects, the solid component can also include bisphenol A (BPA). In such aspects, the BPA can be present in an amount of about 1000 ppm or less, or about 500 ppm or less, or of from about 1 ppm to about 1000 ppm, or of from about 1 ppm to about 500 ppm.

The processes described herein, e.g., the glycolysis and/or alcoholysis processes are substantially mild compared to certain conventional processes, e.g., high temperature one-step glycolysis or methanolysis processes. For instance, certain conventional one-step processes may utilize a glycolysis process at temperatures of 240° C. or above in the presence of a Lewis acid catalyst, for instance, Zn(OAc)₂ or KOAc. Such harsh conditions can result in reduced EG yield from the depolymerization, as the EG is converted in various side reactions to various impurity compounds, including but not limited to: diethylene glycol (DEG), triethylene glycol (TEG), acetaldehyde, 1,1-dimethoxyethane, 1,2-dimethoxyethane, dioxane, 2-methoxyethanol, 1-methoxyethanol, and dimethyl ether. In aspects, the processes described herein are substantially milder than such conventional processes, and also result in less EG yield loss, e.g., from less side reactions converting EG into various impurities. In one aspect, the processes described herein result in about 5% or less yield loss of EG, about 2% or less yield loss of EG, or about 1% or less yield loss of EG, or about 0.5% or less yield loss of EG. In such aspects, the yield loss of EG is the percent of EG that is formed into impurities, e.g., DEG, relative to the combined amount of EG from the polyester composition feed and of the EG added in the glycolysis process. In the same or alternative aspects, the processes described herein result in minimal glycol impurities being produced. For instance, in one aspect, the processes described herein can result in the net generation of about 5 wt. % or less DEG, about 2 wt. % or less DEG, or about 1 wt. % or less DEG, or about 0.5 wt. % or less DEG, or of from about 0.01 wt. % to about 5 wt. % DEG, about 0.01 wt. % to about 2 wt. % DEG, or about 0.01 wt. % to about 1 wt. % DEG, or about 0.01 wt. % to about 0.5 wt. % DEG, or about 0.01 wt. % to about 0.2 wt. % DEG, when EG is used as the one or more glycols in the glycolysis process. In aspects, the processes described herein can result in the net generation of about 5 wt. % or less DEG and/or other impurity, about 2 wt. % or less DEG and/or other impurity, or about 1 wt. % or less DEG and/or other impurity, or about 0.5 wt. % or less DEG and/or other impurity, or of from about 0.01 wt. % to about 5 wt. % DEG and/or other 1 impurity, about 0.01 wt. % to about 2 wt. % DEG and/or other impurity, or about 0.01 wt. % to about 1 wt. % DEG and/or other impurity, or about 0.01 wt. % to about 0.5 wt. % DEG and/or other impurity, or about 0.01 wt. % to about 0.2 wt. % DEG and/or other impurity, when EG is used as the one or more glycols in the glycolysis process. In aspects, the net generation of DEG (or other impurity) is the weight percent of the amount of DEG or other impurity that is present over the amount of DEG or other impurity present in the polyester composition feed. In one aspect, the DEG being produced can be produced in the glycolysis process described herein and/or the alcoholysis process described herein. In certain aspects, the EG and/or any glycol impurities, such as DEG when using EG as the one or more glycols in the glycolysis process, can be present in the resulting liquid component from this alcoholysis step. In certain aspects, utilization of a Lewis base catalyst, e.g., a hydroxide-based or carbonate-based catalyst, in the glycolysis process may also facilitate or contribute to reduced EG degradation and/or a reduction of glycol impurities.

### Recycling Glycols

As discussed above, in various aspects, the glycols utilized in the glycolysis process can be re-used in subsequent rounds of processes for recovery of one or more dialkyl terephthalates disclosed herein. At a high level, in aspects, the liquid component resulting from the alcoholysis process can be processed for re-use, e.g., for re-use in subsequent rounds of glycolysis of a subsequent polyester composition to recover one or more dialkyl terephthalates.

In aspects, as discussed above, the liquid component resulting from the alcoholysis process can include glycols, the alcohol composition, DEG, CHDM, or a combination thereof. In aspects, the glycols in this liquid component can be the glycols that were utilized in the glycolysis process and present with the one or more depolymerization products at the initiation of the alcoholysis process. In various aspects, this liquid component can be subjected to a separation process, e.g., to remove or separate at least a portion of the alcohol composition, for instance, methanol, or a mixture of methanol and ethylene glycol. In certain aspects, for removal of at least a portion of the alcohol composition, the liquid component can be exposed to distillation or short path distillation. In such aspects, the distillation conditions can include exposing the liquid component to a temperature of about 220° C. or less, about 200° C. or less, about 180° C. or less, about 160° C. or less, about 150° C. or less, about 130° C. or less, about 60° C. or more, about 70° C. or more, of from about 60° C. to about 220° C., of from about 70° C. to about 220° C., of from about 60° C. to about 180° C., or of from about 60° C. to about 160° C. In the same or alternative aspects, the distillation conditions can include a pressure of from about 1 Torr (133.3 Pa) to about 800 Torr (106,657 Pa), or about 30 Torr (3999 Pa) to about 500 Torr (66,661 Pa). In aspects, the liquid component can be exposed to the distillation conditions until all or a substantial portion of the alcohol composition has been removed, e.g., vaporized, from the liquid component. In certain aspects, at least a portion of the alcohol composition, if present with the recycle glycols, may be removed during a subsequent glycolysis process, e.g., may be removed or vaporized due to the glycolysis conditions.

In aspects, the distillation of the liquid component can occur in any vessel or distillation system that is suitable for use in the processes and systems described herein. In one aspect, the distillation vessel can be in fluid communication with the alcoholysis reaction vessel and/or any component of the filtering process utilized subsequent to the alcoholysis, e.g., to isolate the dialkyl terephthalate solid or insoluble component. In the same or alternative aspects, the distillation vessel can be in fluid communication with the glycolysis vessel.

In various aspects, the distillation of the liquid component can cause the alcohol composition to vaporize leaving a pot residue. In aspects, the pot residue includes the glycols and any other heavies, e.g., non-vaporizable compounds present in the liquid component. In aspects, the glycols in the pot residue can be referred to as recycle glycols and/or the glycols from a non-vaporizable portion of a continuous distillation process using the distillation conditions described herein can be referred to as recycle glycols.

In aspects, as discussed above, the recycle glycols can be utilized in a subsequent round of the process described herein to recover one or more dialkyl terephthalates from a polyester composition. Further, in aspects, the recycle glycols can be recycled again using the process described herein, after going through this subsequent round of dialkyl terephthalate recovery. In aspects, the recycle glycols can be recovered and re-used at least two, at least three, at least four, or at least five times. In certain aspects, when the recycle glycols are used in subsequent round(s) of dialkyl terephthalate recovery, addition of a catalyst in the subsequent glycolysis step(s) may be omitted, as the recycle glycols may include prior-used catalyst.

In aspects, when the recycles glycols are recovered and re-used, it has been unexpectedly found that the resulting dialkyl terephthalates recovered exhibit comparable purity to that of dialkyl terephthalates recovered using glycols that have not been recovered and re-used. This comparable purity of the dialkyl terephthalate is present, in aspects, after re-using recycle glycols at least two times, at least three times, at least four times, or at least five times resulting in a dialkyl terephthalate recovery having a purity of at least about 90%, at least about 93%, or at least about 95%.

As discussed above, in aspects, the processes described herein are substantially milder than certain conventional processes, and also result in less EG yield loss, e.g., due to fewer side reactions converting EG into various impurities. In one example with three EG recycle experiments, the yield loss of EG to DEG is about 5% or less, about 2% or less, about 1% or less, or about 0.5% or less. In one example with four EG recycle experiments, the yield loss of EG to DEG is about 5% or less, about 2% or less, about 1% or less, or about 0.5% or less. In the same or alternative aspects, the processes described herein result in minimal glycol impurities being produced. In certain aspects, the EG and/or any glycol impurities, such as DEG when using EG as the one or more glycols in the glycolysis process, can be present in the resulting liquid component from the alcoholysis process discussed above. In such aspects, the DEG or any glycol impurities can be recovered and/or present in the recycle glycols described herein. In such aspects, the recycle glycols can include about 5 wt. % or less DEG and/or other impurity, about 2 wt. % or less DEG and/or other impurity, or about 1 wt. % or less DEG and/or other impurity, or about 0.5 wt. % or less DEG and/or other impurity, or of from about 0.01 wt. % to about 5 wt. % DEG and/or other impurity, about 0.01 wt. % to about 2 wt. % DEG and/or other impurity, or about 0.01 wt. % to about 1 wt. % DEG and/or other impurity, or about 0.01 wt. % to about 0.5 wt. % DEG and/or other impurity, or about 0.01 wt. % to about 0.2 wt. % DEG and/or other impurity, when EG is used as the one or more glycols in the glycolysis process.

### Use of Recovered Dialkyl Terephthalates to Form Polyesters or Other Products

As discussed above, the processes disclosed herein can result in high purity dialkyl terephthalates, such as DMT. For instance, in certain aspects, the recovered DMT can be utilized to form one or more polyesters, including but not limited to PET and TMCD-containing polyesters. In certain aspects, the polyesters formed using recovered DMT can be termed renewal polyesters. In various aspects, the products formed using the recovered DMT may be indistinguishable from similar products formed from virgin DMT. In such aspects, any suitable process for forming the PET and TMCD-containing polyesters can be utilized, since the DMT is of sufficient purity.

In the same or alternative aspects, the recovered DMT can be utilized to form CHDM. In various aspects, the CHDM formed using recovered DMT may be indistinguishable from CHDM formed from virgin DMT, due to the high purity of the recovered DMT. In such aspects, the CHDM can be formed from the recovered DMT using any suitable process.

In various aspects, the recovered DMT can be utilized to form one or more plasticizers. In certain aspects, the plasticizers formed using recovered DMT can include dibutyl terephthalate (DBT) and/or dioctyl terephthalate (DOTP). In various aspects, the DBT and/or DOTP formed using recovered DMT may be indistinguishable from the DBT and/or DOTP, respectively, formed from virgin DMT, due to the high purity of the recovered DMT. In such aspects, the DBT and/or DOTP can be formed from the recovered DMT using any suitable process.

### Example Systems

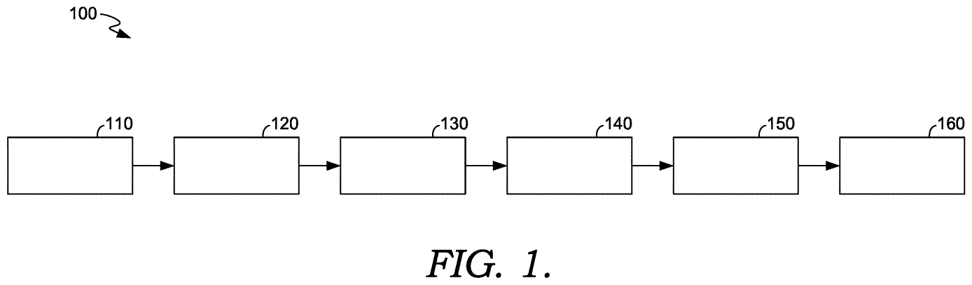

FIG. 1 schematically depicts one example system and/or process for recovering one or more dialkyl terephthalates from a polyester composition. The system 100 includes a source 110 of polyester composition, e.g., the polyester composition described above. Optionally, as described above, the polyester composition can be subjected to a pretreatment step to remove at least a portion of the foreign materials prior to entering the glycolysis and alcoholysis process. process. The vessel 120 represents the glycolysis vessel, where the polyester composition is received and exposed to one or more glycols under depolymerization conditions, as discussed in detail above. In aspects, the vessel 120 can be in fluid communication with the source 110. In various aspects, as discussed above, the polyester composition, after exposure to the depolymerization conditions in the vessel 120, is converted into one or more depolymerization products. In various aspects, as discussed above, the one or more depolymerization products can include monomers and/or oligomers having a degree of polymerization of from 2 to 10, 2 to 8, 2 to 6, or 2 to 4. In aspects, the one or more depolymerization products are present in a mixture that includes a liquid component and a solid component, with the one or more depolymerization products in the liquid component. In aspects, as discussed above, this mixture is exposed to a solid-liquid separation device 130, e.g., a filtering system, where the liquid component, containing the one or more depolymerization products, is separated from the solid component. In various aspects, as discussed herein, the solid-liquid separation device 130 can be in fluid communication with the vessel 120 and/or with the vessel 140. In the aspect depicted in FIG. 1, the one or more depolymerization products and/or the liquid component can be exposed to alcoholysis conditions in a vessel 140. In aspects, the one or more depolymerization products and/or the liquid component can be directly utilized in this alcoholysis process. In such an aspect, the one or more depolymerization products and/or the liquid component may not be subjected to any further processing, e.g., distillation and/or other separation processes, prior to being utilized in this alcoholysis process. Alcoholysis conditions are discussed in detail above. In aspects, as discussed above, the alcoholysis of the one or more depolymerization products and/or the liquid component can result in a mixture that includes an insoluble or solid component that comprises the dialkyl terephthalate and a liquid component that comprises the alcohol composition, glycols, and potentially other soluble components as described herein. As discussed above, the resulting alcoholysis reaction mixture can be exposed to a solid-liquid separation device 150, e.g., a filtering system, to separate the solid component containing the recovered dialkyl terephthalate 160. In aspects, the solid-liquid separation device 150 can be in fluid communication with the vessel 140. In certain aspects, the process described herein associated with the system 100 can be performed as a continuous process, a batch process, or a semi-continuous process. It is understood that the system 100 is just one example system and other configurations of system components are contemplated by the disclosure herein. For instance, one or more of the components of the system 100 may not be physically separated, or distinct, from one or more other components of the system 100. It is further understood that the system 100 is only schematically depicted in order to highlight aspects of the processes disclosed herein.

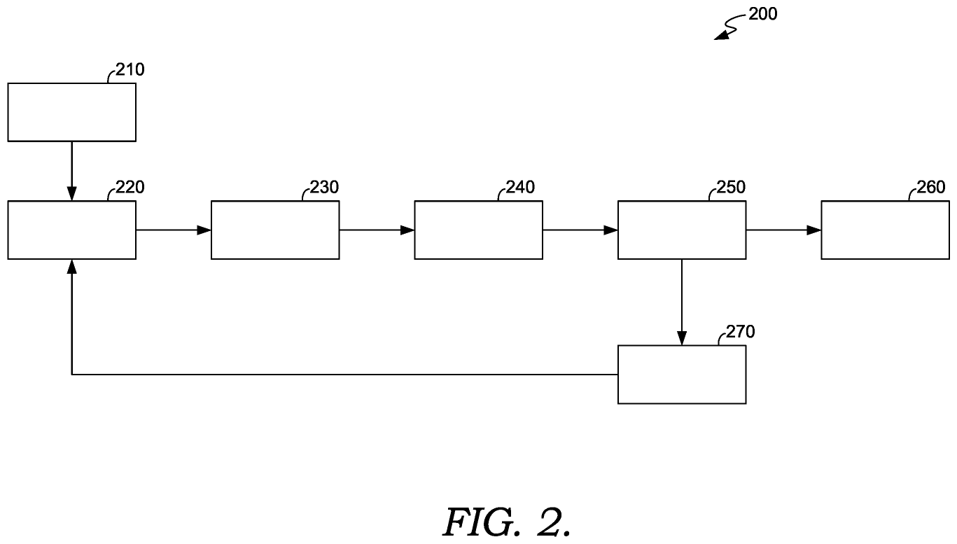

FIG. 2 schematically depicts another example system and/or process for recovering one or more dialkyl terephthalates from a polyester composition. The system 200 includes a source 210 of polyester composition, e.g., the polyester composition described above. Optionally, as described above, the polyester composition can be subjected to a pretreatment step to remove at least a portion of the foreign materials prior to entering the glycolysis and alcoholysis process. The vessel 220 represents the glycolysis vessel, where the polyester composition is received and exposed to one or more glycols under depolymerization conditions, as discussed in detail above. In aspects, the vessel 220 can be in fluid communication with the source 210. In various aspects, as discussed above, the polyester composition, after exposure to the depolymerization conditions in the vessel 220, is converted into one or more depolymerization products. In various aspects, as discussed above, the one or more depolymerization products can include monomers and/or oligomers having a degree of polymerization of from 2 to 10, 2 to 8, 2 to 6, or 2 to 4. In aspects, the one or more depolymerization products are present in a mixture that includes a liquid component and a solid component. In aspects, as discussed above, this mixture is exposed to a solid-liquid separation device 230, e.g., a filtering system, where the liquid component, containing the one or more depolymerization products, is separated from the solid component. In various aspects, as discussed herein, the solid-liquid separation device 230 can be in fluid communication with the vessel 220 and/or with the vessel 240. In the aspect depicted in FIG. 2, the one or more depolymerization products and/or the liquid component can be exposed to alcoholysis conditions in a vessel 240. In aspects, the one or more depolymerization products and/or the liquid component can be directly utilized in this alcoholysis process. In such an aspect, the one or more depolymerization products and/or the liquid component may not be subjected to any further processing, e.g., distillation and/or other separation processes, prior to being utilized in this alcoholysis process. Alcoholysis conditions are discussed in detail above. In aspects, as discussed above, the alcoholysis of the one or more depolymerization products and/or the liquid component can result in a mixture that includes an insoluble or solid component that comprises the dialkyl terephthalate and a liquid component that comprises the alcohol composition, glycols, and potentially other soluble components as described herein. As discussed above, the resulting alcoholysis reaction mixture can be exposed to a solid-liquid separation device 250, e.g., a filtering system, to separate the solid component containing the recovered dialkyl terephthalate 260. In aspects, the solid-liquid separation device 250 can be in fluid communication with the vessel 240. In the aspect depicted in FIG. 2, the system 200 can also include a distillation component 270 for the liquid component resulting from the alcoholysis reaction and separated from the recovered dialkyl terephthalate. In aspects, as discussed above, this liquid component can be exposed to distillation conditions to separate the alcohol composition from the glycols, e.g., at the distillation component 270. The resulting recycle glycols, e.g., in the pot reside post distillation or continuous distillation, can be utilized in the vessel 220 for a subsequent round of dialkyl terephthalate recovery. In various aspects, the distillation component 270 may be in fluid communication with the vessel 220 and/or the solid-liquid separation device 250. In certain aspects not depicted in the figures, optionally, the separated alcohol composition can be processed and returned for re-use in the vessel 240 for subsequent alcoholysis reactions. In certain aspects, the process described herein associated with the system 200 can be performed as a continuous process, a batch process, or a semi-continuous process. It is understood that the system 200 is just one example system and other configurations of system components are contemplated by the disclosure herein. For instance, one or more of the components of the system 200 may not be physically separated, or distinct, from one or more other components of the system 200. It is further understood that the system 200 is only schematically depicted in order to highlight aspects of the processes disclosed herein.

The present disclosure can be further illustrated by the following examples of aspects thereof, although it will be understood that these examples are included merely for purposes of illustration and are not intended to limit the scope of the disclosure unless otherwise specifically indicated.

### EXAMPLES

### Materials

FDST-3 contains 97.7 mole % TPA, 2.3 mole % IPA, 96.7 mole % EG, and 3.3 mole % DEG, and is available from PolyQuest. IV: 0.563 dL/g.

FDST-5 contains 100 mole % TPA, 93.0 mole % EG, 4.1 mole % CHDM, and 2.9 mole % DEG, and is available from Eastman. IV: 0.751 dL/g.

PETG contains 100 mole % TPA, 69 mole % EG, and 31 mole % CHDM, and is available from Eastman. IV: 0.749 dL/g.

Ethylene glycol, methanol, potassium carbonate and 50% sodium hydroxide aqueous solution were obtained from Sigma Aldrich. Nylon-6 (pellet, 3 mm size), nylon-6,6 and polyvinyl chloride (average Mn˜4700) were obtained from Sigma Aldrich. Spandex yarn was obtained from Invista. Polycarbonate (Makrolon® 2658) was obtained from Covestro. Polymethyl methacrylate (PMMA) was obtained from Sumipex and polypropylene (PP3315) was obtained from ExxonMobil. All chemicals and reagents were used as received, unless otherwise mentioned.

### Analytical Procedures

Combustion Ion Chromatography (CIC) Analysis. CIC analysis was carried out using 930 Metrohm Combustion Ion Chromatography system. The sample was first combusted in the Combustion Module at 1000° C. The resulting gases generated during the combustion were dissolved into an absorber solution in the Metrohm 920 Absorber Module. The absorption solution is pre-concentrated in an ion chromatograph and analyzed.

Gas Chromatography (GC) Analysis. GC analysis was performed on an Agilent model 7890B gas chromatograph equipped with a 7693A autosampler and two G4513A towers. The gas chromatograph (GC) was outfitted with two columns—a 60 m×0.32 mm×1.0 micron DB-1701™ (J&W 123-0763) and a 60 m×0.32×1 micron DB-1™ (J&W 123-1063)—and samples were injected simultaneously onto both columns. A shared oven temperature program was used, and sample components were detected by flame ionization detection (FID). Five-point calibrations were performed for components of interest. The gas chromatograph was interfaced to an EZChrom Elite Chromatography Data System.

Methanolysis product samples were prepared by adding a known volume of pyridine-based internal standard solution to a known mass of sample and then derivatizing with N,O-Bis(trimethylsilyl)trifluoroacetamide (BSTFA).

BHET GC yield % was calculated as: (weight % of BHET in crude product by GC)/(theoretical weight % of BHET based on PET/EG charge)*100%

DMT GC yield % was calculated as: (weight of final DMT)/(theoretical DMT weight)*100%.

DMT GC purity % was calculated as: (weight % of DMT in final product by GC)/(total wt. % by GC)*100%. Major impurities shown in GC include MeOH, water and EG. DMT purity for almost every example is greater than 99% if excluding MeOH, water and EG.

DEG (g) in filtrate/wash was calculated as: ((weight of filtrate/wash)×(DEG wt. % by GC/total wt. % by GC)).

Net DEG (g) generated was calculated as: (weight of DEG)−(weight of DEG from polyester composition feed).

Yield loss % of EG to DEG was calculated as: (Net DEG generated)/(EG from polyester composition feed+EG added in the glycolysis process)*100% CHDM (g) in filtrate/wash was calculated as: ((weight of filtrate/wash)*(CHDM wt. % by GC/total wt. % by GC)).

DMI (g) in filtrate/wash was calculated as: (filtrate weight×DMI wt. % by GC/total wt. % by GC+wash weight×DMI wt. % by GC/total wt. % by GC).

Gel Permeation Chromatography (GPC). Size exclusion chromatography GPC analysis was performed on an Agilent series 1100 GPC/SEC analysis system with a UV-Vis detector. The column set used was Polymer Laboratories 5 μm Plgel, with guard, mixed C and oligopore. The eluent consists of 95% methylene chloride and 5% hexafluoroisopropanol with tetraethylammonium nitrate (1 gram/2 liter solvent). The testing was performed at ambient temperature with a flow rate of 1.0 mL/min. Instrument was calibrated with linear PET oligomer standard.

Sample was prepared by dissolving 10 mg sample in 10 mL methylene chloride/hexafluoroisopropanol (70/30). 10 μL Toluene was added as flow rate marker. The injection volume was 10 μL.

Liquid Chromatography (LC). LC analysis for oligomers was performed on an HP 1100 series liquid chromatograph equipped with diode array detector (DAD) with a range of 190-900 nm. The system was fitted with a Zorbax Poroshell 120 EC-C18 (4.6×50 mm, 2.7 μm) column at 40° C. The flow rate was 1.0 mL/min. Mobile phases were water (25 nM ammonium acetate) (A) and acetonitrile (B). The elution gradient was as follows: 0 min, 95% A/5% B; 2 min, 95% A/5% B; 18 min, 0% A/100% B; 28 min, 0% A/100% B; 28.1 min, 95% A/5% B; 33 min, 95% A/5% B. Sample solution was prepared by dissolving ˜4 mg sample in 1 mL DMF/DMSO (50/50). The injection volume was 2 μL. Oligomer distribution was reported as area %.

LC analysis for BPA was performed on an HP 1100 series liquid chromatograph equipped with a fluorescence detector using excitation wavelength of 225 nm, emission wavelength of 310 nm, a FLD PMT gain of 10 and data frequency of 2.31 Hz. The system was fitted with an Agilent Poroshell EC-C18 (4.6×150 mm, 2.7 μm) column at 30° C. Mobile phases were 0.14% phosphoric acid in water (A), acetonitrile (B) and THF (C). The elution gradient was as follows: 0 min, 79% A/0% B/21% C; 10 min, 79% A/0% B/21% C; 18 min, 34% A/45% B/21% C; 18.1 min, 14% A/65% B/21% C; 19 min, 14% A/65% B/21% C; 19.1 min, 79% A/0% B/21% C; 25 min, 79% A/0% B/21% C. The flow rate was 0.9 mL/min. BPA content was reported ppm (μg/g).

$$Theoretical BPA level was calculated as : ( ( polycarbonate ( PC ) weight / \\ PC unit molecular weight ) * BPA molecular weight ) / ( theoretical DMT weight ) * 1000000$$

Inherent Viscosity Measurement. The Inherent viscosities (IV) of the particular polymer materials useful herein are determined according to ASTM D2857-70 procedure, in a Wagner Viscometer of Lab Glass, Inc., having a ½ mL capillary bulb, using a polymer concentration about 0.5% by weight in 60/40 by weight of phenol/tetrachloroethane. The procedure is carried out by heating the polymer/solvent system at 120° C. for 15 minutes, cooling the solution to 25° C. and measuring the time of flow at 25° C. The IV is calculated from the equation:

$${η}_{inh} = \frac{ln \frac{{t}_{S}}{{t}_{0}}}{C}$$

where: η: inherent viscosity at 25° C. at a polymer concentration of 0.5 g/100 mL of solvent; tS: sample flow time; t0: solvent-blank flow time; C: concentration of polymer in grams per 100 mL of solvent. The units of the inherent viscosity throughout this application are in the deciliters/gram.

In the following examples, a viscosity was measured in tetrachloroethane/phenol (50/50, weight ratio) at 30° C. and calculated in accordance with the following equation:

$${η}_{inh} = \frac{ln ( {η}_{s p} )}{C}$$

wherein ηₛₚ is a specific viscosity and C is a concentration.

### Example 1: Glycolysis Catalysts and Temperature Studies

In this Example 1, PET (3 g) and EG (7 g) and catalyst were charged into a 20-mL vial with a magnetic stirring bar. The resulting mixture was heated in a heating block at a set temperature and stirred for 4 hours. Reaction aliquot was analyzed by GC analysis. Various catalysts were utilized at various temperatures for PET glycolysis (Table 1). Bis 2-Hydroxyethyl Terephthalate (BHET) yield, e.g., BHET wt. % was determined using GC analysis. BHET wt. % is relative to the total weight of the PET in the glycolysis reaction. The results appear in Table 1. As can be seen in Table 1, a number of glycolysis catalysts exhibited high (e.g., 40% and above) BHET GC yield at a wide variety of glycolysis temperatures.

TABLE 1
Glycolysis Catalysts and Glycolysis Temperatures

| Experiment | | Temperature | BHET (GC |
|---|---|---|---|
| Reference | Catalyst | (° C.) | yield %) |
| 1A | K₂CO₃ | 150 | 60.2% |
| 1B | Na₂CO₃ | 150 | 46.9% |
| 1C | LiOH | 150 | 8.7% |
| 1D | NaOH | 150 | 12.2% |
| 1E | KOH | 150 | 5.1% |
| 1F | tetra isopropyl titanate | 150 | 2.2% |
| | (TIPT) | | |
| 1G | butyltin tris-2- | 150 | 18.3% |
| | ethylhexanoate (FASCAT | | |
| | 4102) | | |
| 1H | ZrCO₃ | 150 | 0.8% |
| 1I | 1,8- | 150 | 66.2% |
| | Diazabicyclo[5.4.0]undec- | | |
| | 7-ene (DBU) | | |
| 1J | NaOMe | 150 | 49.5% |
| 1K | LiOMe (2M) | 150 | 39.1% |
| 1L | zinc acetylacetonate | 150 | 40.3% |
| | hydrate (Zn(acac)₂) | | |
| 1M | zinc acetate (Zn(OAc)₂) | 150 | 23.2% |
| 1N | tetrabutylammonium | 150 | 13.3% |
| | bromide (TBAH) | | |
| 1O | K₂CO₃ | 170 | 69.0% |
| 1P | Na₂CO₃ | 170 | 68.6% |
| 1Q | LiOH | 170 | 60.4% |
| 1R | NaOH | 170 | 66.2% |
| 1S | KOH | 170 | 61.0% |
| 1T | KOH | 170 | 61.0% |
| 1U | TIPT | 170 | 44.0% |
| 1V | FASCAT 4102 | 170 | 71.8% |
| 1W | ZrCO₃ | 170 | 2.6% |
| 1X | DBU | 170 | 69.1% |
| 1Y | NaOMe | 170 | 72.0% |
| 1Z | K₂CO₃ | 190 | 69.2% |
| 1AA | Na₂CO₃ | 190 | 70.6% |
| 1AB | LiOH | 190 | 79.3% |
| 1AC | NaOH | 190 | 80.5% |
| 1AD | KOH | 190 | 77.3% |
| 1AE | TIPT | 190 | 79.6% |
| 1AF | FASCAT 4102 | 190 | 75.2% |
| 1AG | ZrCO₃ | 190 | 16.7% |
| 1AH | DBU | 190 | 67.4% |
| 1AI | NaOMe | 190 | 75.0% |
| 1AJ | LiOMe | 190 | 67.1% |
| 1AK | Zn(acac)₂ | 190 | 69.6% |
| 1AL | TBAH | 190 | 74.7% |
| 1AM | ZrCO₃ | 190 | 20.2% |
| 1AN | CaCO₃ | 200 | 8.7% |
| 1AO | K₂CO₃ | 180 | 63.6% |
| 1AP | K₂CO₃ | 200 | 91.4% |
| 1AQ | Mg(OMe)₂ | 190 | 72.6% |
| 1AR | Zn(acac)₂/LiCl | 190 | 51.4% |
| 1AS | Zn(OAc)₂/NaOMe | 190 | 61.2% |
| 1AT | Mn(OAc)₂ | 195 | 83.2% |
| 1AU | Hydrotalcite | 200 | 88.2% |

Several glycolysis products from catalyst study were analyzed by LC and GC analysis. The result is summarized in Table 1B. Different catalysts delivered a similar level of degree of polymerization (e.g., 1AP, 1AT and 1AU). Hydrotalcite may be interest in certain instances, such as in the use of fixed-bed reactor in the depolymerization of the polyester composition.

TABLE 1B
The impact of catalyst on oligomer distribution

| | | LC (area %) | | | |
|---|---|---|---|---|---|
| Experiment | | Monomer % | | | GC (wt %) * |
| Reference | Catalyst | (BHET %) | Dimer % | ≥Trimer % | BHET yield % |
| Example 1AP | K₂CO₃ | 78.9% | 18.2% | 2.9% | 91.4% |
| Example 1AT | Mn(OAc)₂ | 75.1% | 17.5% | 7.4% | 83.2% |
| Example 1AN | CaCO₃ | 43.4% | 26.7% | 29.9% | 8.7% |
| Example 1AU | Hydrotalcite | 78.8% | 18.3% | 2.9% | 88.2% |

* Yield was calculated based on GC analysis.

### Examples 2A-2D: Examples of Glycolysis/Methanolysis Reactions at Varying PET Wt. % in EG

### Example 2A: 70 wt. % PET in EG

A 3-necked 1-liter round-bottom flask was equipped with a mechanical stirrer, a reflux condenser and a thermocouple. Charge FDST-5 pellet (350.24 g), potassium carbonate (3.51 g) and ethylene glycol (151.3 g). The resulting mixture was heated to 200° C. under nitrogen atmosphere and hold at 200° C. until PET pellet dissolved. The heating mantle was removed, and the solution was allowed to cool to 75° C. Crude mixture was poured into a sample jar. Crude product was collected as white wax solid (495.82 g). Methanolysis was carried out with 100 g crude product.

A 3-necked 500-mL round bottomed flask was further equipped with a mechanical stirrer, a reflux condenser and a thermocouple. Charge 100 g crude product and MeOH (280.21 g). The resulting mixture was heated to 60° C. before 50% NaOH solution (0.353 mL) was added dropwise. Stirring was continued for 15 min. Remove the heating mantle and allow the flask to cool to room temperature for 2 hours. The reaction mixture was further cooled in an ice bath. Product was recovered by filtration/MeOH wash and dried in the air. Product was obtained as white crystalline solid (65.05 g, 93% yield, 97% GC purity).

### Example 2B: 50 wt. % PET in EG

A 3-necked 1-liter round-bottom flask was equipped with a mechanical stirrer, a reflux condenser and a thermocouple. Charge FDST-5 pellet (100.02 g), potassium carbonate (0.97 g) and ethylene glycol (99.91 g). The resulting mixture was heated to 195° C. under nitrogen atmosphere and hold at 195° C. until PET pellet dissolved. The heating mantle was removed, and the solution was allowed to cool to ambient temperature. To the mixture, charge methanol (403.89 g). The resulting mixture was heated to 60° C. before 50% NaOH solution (0.416 mL) was added dropwise. Stirring was continued for 15 min. Remove the heating mantle and allow the flask to cool to room temperature for 2 hours. The reaction mixture was further cooled in an ice bath. Product was recovered by filtration/wash and dried in the air. DMT Product was obtained as white crystalline solid (82.82 g, 82% yield, 99% GC purity).

### Example 2C: 33 wt. % PET in EG, NaOH Catalysts for Glycolysis and Methanolysis Steps

A 3-necked 1-liter round-bottom flask was equipped with a mechanical stirrer, a reflux condenser and a thermocouple. Charge FDST-5 pellet (75.10 g), 50% NaOH aqueous solution (0.312 mL) and ethylene glycol (150.57 g). The resulting mixture was heated to 195° C. under nitrogen atmosphere and hold at 195° C. until PET pellet. The heating mantle was removed, and the solution was allowed to cool to ambient temperature. To the mixture, charge methanol (302.13 g). The resulting mixture was heated to 60° C. before 50% NaOH solution (0.312 mL) was added dropwise. Stirring was continued for 15 min. Remove the heating mantle and allow the flask to cool to room temperature for 2 hours. The reaction mixture was further cooled in an ice bath. Product was recovered by filtration/wash and dried in the air. DMT Product was obtained as white crystalline solid (67.61 g, 89.2% yield, 98.4% GC purity).

### Example 2D: 30 wt. % PET in EG, and 60 wt. % PET in EG

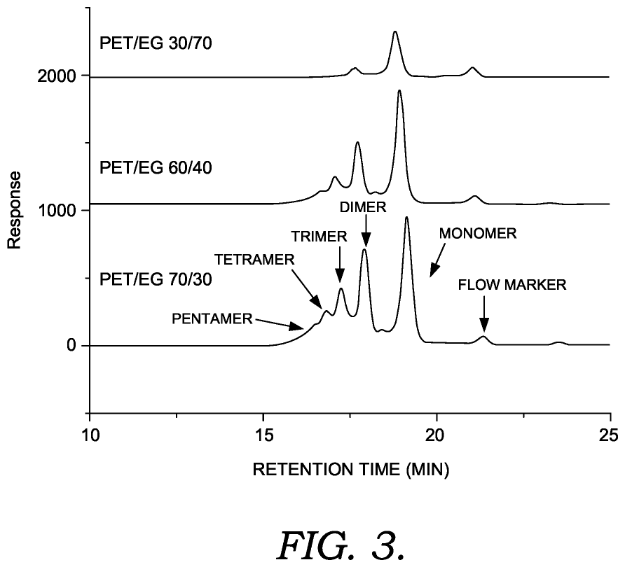

Additional example glycolysis and methanolysis reactions were carried out on a 30 wt. % PET in EG, and on a 60 wt. % PET in EG at the same catalyst loading under conditions as described above in Examples 2A-2C. GPC and GC analyses were performed on a portion of the resulting mixture prior to additional of methanol, as well as the same mixture from Example 2A to discern the oligomeric distribution of the product. The results are provided below in Table 2 and in FIG. 3. FIG. 3 depicts the GPC curve at 255 nanometers (nm).

TABLE 2
The Impact of PET wt. % on Oligomer Distribution

| | | | | | GC |
|---|---|---|---|---|---|
| | | GPC (area %) | | | (wt %) * |
| | PET/EG | Monomer % | Dimer | ≥Trimer | BHET |
| Experiment | (by wt) | (BHET %) | % | % | yield % |
| Example 2D | 30/70 | 76.2% | 15.3% | 8.6% | 79.1% |
| Example 2D | 60/40 | 49.4% | 25.1% | 25.6% | 48.1% |
| Example 2A | 70/30 | 36.4% | 25.0% | 37.7% | 41.9% |

* Yield was calculated based on GC analysis.

As can be seen in Table 2 and in FIG. 3, the PET/EG ratio can affect the oligomeric distribution of the glycolysis products.

### Example 3: Recovery of DMT from Copolyester PETG

A 3-necked 1-liter round-bottom flask was equipped with a mechanical stirrer, a reflux condenser and a thermocouple. PETG (100.09 g), potassium carbonate (1.01 g), and ethylene glycol (200.59 g) were added to the flask. The resulting mixture was heated to 195° C. under nitrogen atmosphere and held until the PET pellet dissolved. The heating mantle was removed, and the solution was allowed to cool to ambient temperature. To the mixture, methanol (400.39 g) was added. The resulting mixture was heated to 50° C. before 50% NaOH solution (0.625 g) was added dropwise. Stirring was continued for 30 min. The heating mantle was removed and the flask was allowed to cool to room temperature for 2 hours. The reaction mixture was further cooled in an ice bath. Product was recovered by filtration/wash and dried in the air. DMT Product was obtained as white crystalline solid (79.26 g, 88% yield, 98% GC purity).

### Examples 4A-4H: Recovery of DMT from a Mixture of PET and Impurity Polymer

### Example 4A: PET and PVC

A 3-necked 500-mL round-bottom flask was equipped with a mechanical stirrer, a reflux condenser and a thermocouple. Charge FDST-5 pellet (75.0 g), polyvinyl chloride (7.5 g), potassium carbonate (0.75 g) and ethylene glycol (150 g). The resulting mixture was heated to 195° C. and hold at 195° C. for 2 hours under nitrogen atmosphere. The heating mantle was removed, and the solution was allowed to cool to 75° C. Crude mixture was filtered into a 3-necked 1-liter round-bottom flask through a preheated filter funnel. Insoluble solid was washed with methanol (50 g). Insoluble PVC was recovered and dried in the air (7.01 g).

An additional 250 g methanol was added to the filtrate. The 3-necked 1-liter round bottomed flask was further equipped with a mechanical stirrer, a reflux condenser and a thermal couple. The resulting mixture was heated to 60° C. before 50% NaOH solution (0.312 mL) was added dropwise. Stirring was continued for 15 min. Remove the heating mantle and allow the flask to cool to room temperature for 2 hours. The reaction mixture was further cooled in an ice bath. Product was recovered by filtration and the filter cake was washed with cold MeOH (2×150 g). Product was dried in the air to deliver white crystalline solid (67.07 g, 88.5% yield, 97% GC purity). Chloride level in the DMT product was measured to be 48 ppm using combustion ion chromatography (CIC).

### Example 4B: Spandex and PET

The same procedure as in Example 4A was carried out, except Spandex was used instead of PVC. Product (66.33 g) was isolated in 87.5% yield and 95% GC purity.

### Example 4C: Nylon 6-6 and PET

The same procedure as in Example 4A was carried out, except nylon-6,6 was used instead of PVC. Product (66.37 g) was isolated in 87.6% yield and 98% GC purity.

### Example 4D: Poly(Methyl Methacrylate) and PET

The same procedure as in Example 4A was carried out, except poly(methyl methacrylate) was used instead of PVC. Product (70.04 g) was isolated in 92.0% yield and 98% GC purity.

### Example 4E: Polypropylene and PET

The same procedure as in Example 4A was carried out, except polypropylene was used instead of PVC. Product (67.05 g) was isolated in 88.4% yield and 97% GC purity.

### Example 4F: Nylon-6 and PET

The same procedure as in Example 4A was carried out, except nylon-6 was used instead of PVC. Product (68.94 g) was isolated in 91.0% yield and 97% purity.

### Example 4G: Polycarbonate and PET

The same procedure as in Example 4A was carried out, except polycarbonate was used instead of PVC. Product (63.92 g) was isolated in 84.0% yield and 96% GC purity. LC analysis showed that DMT product contained 620 ppm BPA.

### Example 4H: Cotton Cloth and PET

The same procedure as in Example 4A was carried out, except mincing cotton cloth was used instead of PVC. Product (61.31 g) was isolated in 80.8% yield and 99% GC purity.

### Example 5: Methanolysis Conducted at Reflux Temperature

A 3-necked 1-liter round-bottom flask was equipped with a mechanical stirrer, a reflux condenser and a thermocouple. Charge FDST-3 pellet (98.07 g), potassium carbonate (0.98 g) and ethylene glycol (41.93 g). The resulting mixture was heated to 195° C. under nitrogen atmosphere and hold at 195° C. until PET pellet dissolved. The heating mantle was removed, and the solution was allowed to cool to ambient temperature. To the mixture, charge methanol (392.21 g). The resulting mixture was heated to reflux before 50% NaOH solution (0.407 mL) was added dropwise. Stirring was continued for 15 min. Remove the heating mantle and allow the flask to cool to room temperature for 2 hours. The reaction mixture was further cooled in an ice bath. Product was recovered by filtration, washed and dried. DMT Product was obtained as white crystalline solid (91.01 g, 91.8% yield, 99% GC purity).

### Example 6A-6C: Glycolysis Followed by EG Distillation

### Example 6A: 30 wt. % PET in EG

A 3-necked 1-liter round-bottom flask was equipped with a mechanical stirrer, a reflux condenser and a thermocouple. Charge FDST-5 pellet (90.09 g), potassium carbonate (0.92 g) and ethylene glycol (209.14 g). The resulting mixture was heated to 195° C. under nitrogen atmosphere and hold at 195° C. until PET pellet dissolved. The heating mantle was removed, and the solution was allowed to cool to ambient temperature. GC wt % analysis showed that crude product contained 30.04% BHET and 58.18% EG. LC analysis showed a mixture of 79.51% BHET (monomer), 18.08% dimer, 2.24% trimer, and 0.16% 4+ mer by area % LC analysis.

EG distillation was carried out using short-path distillation at 82-87° C. and 5 torr. Collected 150.66 g EG and 138.74 g pot residue. Analysis showed that the pot residue contained 59.98% BHET and 16.85% EG by wt % GC analysis; 70.99% BHET (monomer), 25.18% dimer, 3.52% trimer and 0.30% 4+ mer by area % LC analysis.

### Example 6B: 30 wt. % PET in EG

A 3-necked 1-liter round-bottom flask was equipped with a mechanical stirrer, a reflux condenser and a thermocouple. Charge FDST-5 pellet (90.04 g), potassium carbonate (0.90 g) and ethylene glycol (210.29 g). The resulting mixture was heated to 195° C. under nitrogen atmosphere and hold at 195° C. until PET pellet dissolved. The heating mantle was removed, and the solution was allowed to cool to ambient temperature.

EG distillation was carried out using short-path distillation at 120° C. and 50 torr. Collect 153.15 g EG and 136.32 g pot residue. Analysis showed that the pot residue contained 46.88% BHET and 21.12% EG by wt. % GC analysis; 45.51% BHET (monomer), 34.33% dimer, 15.53% trimer and 3.90% 4+ mer by area % LC analysis.

### Example 6C: 30 wt. % PET in EG

A 3-necked 1-liter round-bottom flask was equipped with a mechanical stirrer, a reflux condenser and a thermocouple. Charge FDST-5 pellet (90.09 g), potassium carbonate (0.89 g) and ethylene glycol (213.13 g). The resulting mixture was heated to 195° C. under nitrogen atmosphere and hold at 195° C. until PET pellet dissolved. The heating mantle was removed, and the solution was allowed to cool to ambient temperature.

EG distillation was carried out using short-path distillation at 145° C. and 150 torr. Collect 150.9 g EG and 149.29 g pot residue. Analysis showed that the pot residue contained 40.73% BHET and 25.31% EG by wt. % GC analysis; 44.13% BHET (monomer), 33.91% dimer, 16.49% trimer and 4.43% 4+ mer by area % LC analysis.

Table 3 below depicts oligomeric distributions of the analyzed samples from Examples 6A-6C mentioned above.

TABLE 3
Impact of EG Distillation on Oligomeric Distribution

| | | Distillation | Monomer | | | |
|---|---|---|---|---|---|---|
| | | condition | area % | Dimer | Trimer | 4-mer |
| Experiment | Description | (° C., torr) | (BHET) | area % | area % | area % |
| Example 5A | Crude product | None | 79.51 | 18.08 | 2.24 | 0.16 |
| | From 30% PET/EG | | | | | |
| Example 5A | EG distillation | 88° C., 5 torr | 70.99 | 25.18 | 3.52 | 0.30 |
| Example 5B | EG distillation | 120° C., 50 torr | 45.51 | 34.33 | 15.53 | 3.90 |
| Example 5C | EG distillation | 145° C., 150 torr | 44.13 | 33.91 | 16.49 | 4.43 |

* Product was analyzed by LC and reported as area %.

Without being bound by any particular theories, the data from Table 3 suggests that the PET depolymerization would be an equilibrium. That is, the formation of low MW oligomer (i.e. monomer) would compete with back reaction to form the high MW oligomer (i.e. 4-mer). The equilibrium constant would be dependent on temperature. The lower temperature (i.e. 88° C.) would not affect the equilibrium (see rows 1 vs. 2—Example 6A), while the higher temperature would significantly shift the equilibrium to the higher MW oligomer (see rows 3 and 4—Example 6B and Example 6C).

### Examples 7A-7D: EG Recycling

### Example 7A

A 3-necked 1-liter round-bottom flask was equipped with a mechanical stirrer, a reflux condenser and a thermocouple. Charge FDST-3 pellet (100.0 g), potassium carbonate (1.02 g) and ethylene glycol (150.0 g). The resulting mixture was heated to 195° C. under nitrogen atmosphere and hold at 195° C. until PET pellet dissolved. The heating mantle was removed, and the mixture was allowed to cool to ambient temperature. To the mixture, charge methanol (301.55 g). The resulting mixture was heated to 50° C. before 50% NaOH solution (0.416 mL) was added dropwise. Stirring was continued for 30 min. Remove the heating mantle and allow the flask to cool to room temperature for 2 hours. The reaction mixture was further cooled in an ice bath. Product was recovered by filtration. Wash the filter cake with MeOH and dry the product in the air. DMT Product was obtained as white crystalline solid (92.46 g, 91% yield, 98% GC purity).

The filtrate and wash were combined and subjected to distillation using short-path distillation at 67.1° C. MeOH (270.69 g) was collected (99% GC purity). The pot residue was obtained (183.5 g), which was used for the PET glycolysis without further purification.

### Example 7B

A 3-necked 1-liter round-bottom flask was equipped with a mechanical stirrer, a reflux condenser and a thermocouple. Charge FDST-3 pellet (100.1 g) and pot residue from Example 6A (183.5 g). The resulting mixture was heated to 195° C. under nitrogen atmosphere and hold at 195° C. until PET pellet dissolved. The heating mantle was removed, and the mixture was allowed to cool to ambient temperature. To the mixture, charge methanol (301.76 g). The resulting mixture was heated to 50° C. before 50% NaOH solution (0.416 mL) was added dropwise. Stirring was continued for 30 min. Remove the heating mantle and allow the flask to cool to room temperature for 2 hours. The reaction mixture was further cooled in an ice bath. Product was recovered by filtration. Wash the filter cake with MeOH and dry the product in the air. DMT Product was obtained as white crystalline solid (97.49 g, 96% yield, 98% GC purity).

The filtrate and wash were combined and subjected to distillation using short-path distillation at 69.3° C. MeOH (269.3 g) was collected (99% GC purity). The pot residue (211.7 g) was used for the PET glycolysis without further purification.

### Example 7C

A 3-necked 1-liter round-bottom flask was equipped with a mechanical stirrer, a reflux condenser and a thermocouple. Charge FDST-3 pellet (100.11 g) and pot residue from Example 6B. The resulting mixture was heated to 195° C. under nitrogen atmosphere and hold at 195° C. until PET pellet dissolved. The heating mantle was removed, and the mixture was allowed to cool to ambient temperature. To the mixture, charge methanol (303.48 g). The resulting mixture was heated to 50° C. before 50% NaOH solution (0.416 mL) was added dropwise. Stirring was continued for 30 min. Remove the heating mantle and allow the flask to cool to room temperature for 2 hours. The reaction mixture was further cooled in an ice bath. Product was recovered by filtration. Wash the filter cake with MeOH and dry the product in the air. DMT Product was obtained as white crystalline solid (98.08 g, 97% yield, 97% GC purity).

The filtrate and wash were combined and subjected to distillation using short-path distillation at 68.6° C. MeOH (282.6 g) was collected (99% GC purity). The pot residue (289.6 g) was used for the PET glycolysis without further purification.

### Example 7D

A 3-necked 1-liter round-bottom flask was equipped with a mechanical stirrer, a reflux condenser and a thermocouple. Charge FDST-3 pellet (100.5 g) and pot residue from Example 6C. The resulting mixture was heated to 195° C. under nitrogen atmosphere and hold at 195° C. until PET pellet dissolved. The heating mantle was removed, and the mixture was allowed to cool to ambient temperature. To the mixture, charge methanol (399.4 g). The resulting mixture was heated to 50° C. before 50% NaOH solution (0.416 mL) was added dropwise. Stirring was continued for 30 min. Remove the heating mantle and allow the flask to cool to room temperature for 2 hours. The reaction mixture was further cooled in an ice bath. Product was recovered by filtration. Wash the filter cake with MeOH and dry the product in the air. DMT Product was obtained as white crystalline solid (100.53 g, 99% yield, 96% GC purity).

### Example 8: Comparative Example—Conventional PET Methanolysis

A 100-mL autoclave was charged with FDST-5 pellet (15.0 g), methanol (45.0 g) and zinc acetate (0.15 g). The resulting mixture was heated to 260° C. and stirred for 4 hours. After completion, the autoclave was allowed to cool to ambient temperature. Crude reaction mixture was taken up in dichloromethane (100 mL) and washed with water. The organic layer was dried over sodium sulfate and concentrated. DMT product was obtained as off-white solid (14.1 g, 93.0% yield, 88.2% GC purity).

In this Example 8, a conventional methanolysis process was carried out on PET. This example shows that the above example glycolysis/methanolysis processes can achieve comparable, or better DMT yields, and purity.

### Example 9: Impact of Methanol/PET on the Final Product Quality

In this Example 9, a range of Methanol/PET ratios were evaluated in a 2-step glycolysis and methanolysis process, similar to that performed in Examples 7A-7D. The glycolysis step was carried out at 190° C. and the methanolysis step was run at 60° C. The results appear in Table 4 below.

TABLE 4
Impact of Methanol/PET on the Final Product Quality

| Experiment | MeOH/PET (wt/wt) | DMT Yield % | GC purity % |
|---|---|---|---|
| Example 8A | 4/1 | 91.5% | 99.2% |
| Example 8B | 3/1 | 92.3% | 98.3% |
| Example 8C | 2/1 | 93.3% | 97.6% |

As shown in Table 4, the methanol/PET ratio does not significantly affect the DMT product yield and purity. For instance, up to a 2/1 ratio of MeOH/PET worked well to deliver the DMT product in good yield and purity.

### Example 10: PVC Removal

In this Example 10, various process samples of Example 4A (recovery of DMT from PET/PVC) were analyzed to discern the chloride content. Particularly, the insoluble material post-glycolysis was analyzed, as was the methanolysis filtrate, and the DMT product. Table 5 below lists the results of this analysis.

TABLE 5
Chloride Levels in DMT Product, Methanolysis
Filtrate, and Glycolysis Insoluble Fraction

| Sample | Cl (ppm) | Cl distribution (wt %) |
|---|---|---|
| DMT | 48 | 0.08% |
| Methanolysis filtrate | 531 | 3.93% |
| Recovered Insoluble | too high | 93.6% |
| after glycolysis | | |

As shown in Table 5, 93.6% chloride was recovered in the insoluble form, which exceeds the detection limit of chloride level using CIC analysis. 531 ppm Chloride was measured in the combined filtrate and methanol wash, which accounts for 3.9% chloride in the chloride balance. Only 48 ppm chloride was found in the isolated DMT product.

### Example 11: BPA Removal

In this Example 11, various process samples of Example 4G (recovery of DMT from PET/PVC) were analyzed to discern the bisphenol A (BPA) content. Table 6 below lists the results of this analysis.

TABLE 6
BPA analysis

| | | Theoretical BPA level |
|---|---|---|
| Sample | BPA (ppm) | based on feed (ppm) |
| DMT product | 620 | 83002 |

As shown in Table 6, only 620 ppm BPA was found in the final DMT product after MeOH wash. Without further optimization, our process was demonstrated to remove 99.3% BPA. Without being bound by any particular theory, further optimization of wash process may further reduce the BPA level.

### Example 12: Analysis of Recycling EG for Use in Subsequent DMT Recoveries

In this Example 12, DMT was recovered from PET (FDST-5) in a 2-step partial glycolysis and methanolysis process, similar to that described above in Example 7A, where the methanolysis filtrate was then subjected to short path distillation, as outlined above in Example 7A, and the pot residue was reused as an EG source in a subsequent partial glycolysis and methanolysis process, as in Example 7B. This process was repeated four times so that the pot residue of short path distillation of the methanolysis filtrate was re-used a total of four times (for 2-step partial glycolysis and methanolysis process for DMT recovery without adding in new EG for the glycolysis step). Table 6 below describes this example and also lists the DMT yield and GC purity.

TABLE 7
Recycling EG for Use in Subsequent DMT Recoveries

| | EG | K2CO3 | Aq. NaOH | | |
|---|---|---|---|---|---|
| | charged | charge | (50% Conc.) | DMT | GC |
| | (g) | (g) | charge (g) | Yield % | purity % |
| Example 12A | 70.23 | 0.70 | 0.43 | 90% | 99% |
| Example 12B (re- | 0 | 0.70 | 0.43 | 90% | 98% |
| use of recycled | | | | | |
| EG from Example | | | | | |
| 12A) | | | | | |
| Example 12C (re- | 0 | 0.71 | 0.43 | 95% | 99% |
| use of recycled | | | | | |
| EG from Example | | | | | |
| 12B) | | | | | |
| Example 12D (re- | 0 | 0 | 0.43 | 97% | 98% |
| use of recycled | | | | | |
| EG from Example | | | | | |
| 12C) | | | | | |
| Example 12E (re- | 0 | 0 | 0.43 | 99% | 99% |
| use of recycled | | | | | |
| EG from Example | | | | | |
| 12D) | | | | | |

As can be seen in Table 7, the EG can be recycled and re-used without further purification at least four successive times in DMT recovery processes without affecting DMT product yield and purity.

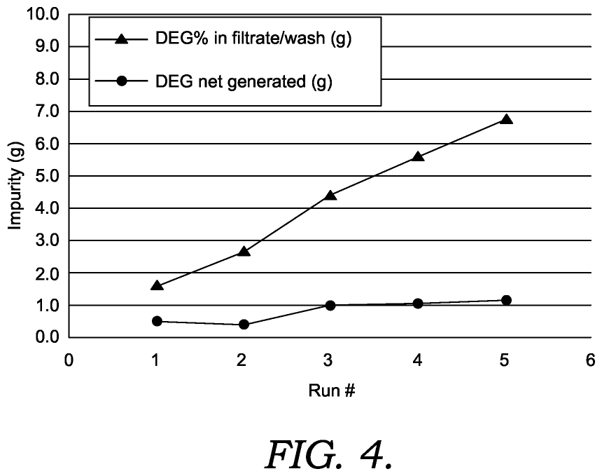

Table 8 below details an impurity distribution from Examples 12A-12E, and FIG. 4 also graphically depicts the data in Table 8.

TABLE 8
Glycol Distribution

Glycol in filtrate and wash

| | | | net DEG | EG yield % |
|---|---|---|---|---|
| | Repeat | DEG | generated | loss to DEG |
| Experiment | run | (g) | (g) | formation |
| Example 12A | 1 | 1.69 | 0.57 | 0.63% |
| Example 12B | 2 | 2.73 | 0.47 | 0.42% |
| (re-use of | | | | |
| recycled EG | | | | |
| from Example | | | | |
| 12A) | | | | |
| Example 12C | 3 | 4.41 | 1.03 | 0.76% |
| (re-use of | | | | |
| recycled EG | | | | |
| from Example | | | | |
| 12B) | | | | |
| Example 12D | 4 | 5.61 | 1.10 | 0.69% |
| (re-use of | | | | |
| recycled EG | | | | |
| from Example | | | | |
| 12C) | | | | |
| Example 12E | 5 | 6.80 | 1.16 | 0.63% |
| (re-use of | | | | |
| recycled EG | | | | |
| from Example | | | | |
| 12D) | | | | |

As can be seen in Table 8 and in FIG. 4, a small amount of DEG was generated in the EG recycle Example 12, using FDST-5. DEG and CHDM stayed in the filtrate and wash.

### Example 13: Analysis of Recycling EG for Use in Subsequent DMT Recoveries

In this Example 13, the product and impurities were analyzed from the EG Recycling experiments of Examples 7A-7D. Table 9 lists the DMT yield, GC purity, and DMI content in the product.

TABLE 9
Recycling EG for Use in Subsequent DMT Recoveries

| | EG | K2CO3 | Aq. NaOH | DMT | GC | DMI |
|---|---|---|---|---|---|---|
| | charged | charge | (50% Conc.) | Yield | purity | % in |
| | (g) | (g) | charge (g) | % | % | product |
| Example 7A | 150 | 1.02 | 0.62 | 91% | 98% | 0% |
| Example 7B | 0 | 0 | 0.62 | 96% | 98% | 0.04% |
| Example 7C | 0 | 0 | 0.62 | 97% | 97% | 0.02% |
| Example 7D | 0 | 0 | 0.62 | 99% | 96% | 0.04% |

As can be seen in Table 9, the EG can be recycled and re-used without further purification at least four successive times in DMT recovery processes without affecting DMT product yield and purity. DMI level in the product stayed low throughout.

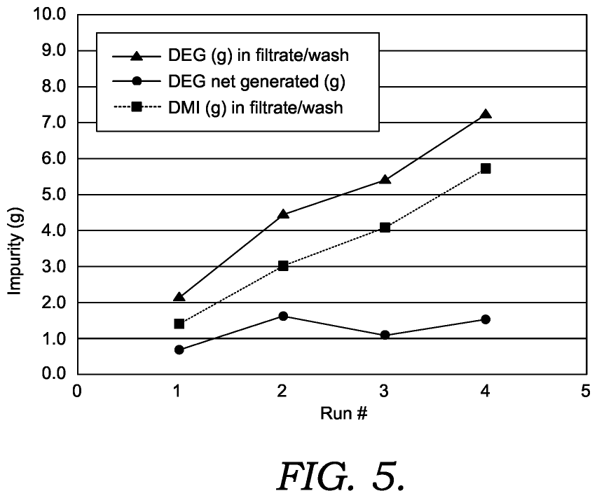

Table 10 below details an impurity distribution for Examples 7A-7D, and FIG. 5 also graphical depicts the data in Table 10.

TABLE 10
Glycol Distribution

| | | Components of interest In filtrate/wash | | | |
|---|---|---|---|---|---|
| | Repeat | DEG | Net DEG | EG yield % loss to | DMI |
| | run | (g) | generated (g) | DEG formation | (g) |
| Example 7A | 1 | 2.08 | 0.65 | 0.36% | 1.39 |
| Example 7B | 2 | 4.44 | 1.58 | 0.75% | 2.96 |
| Example 7C | 3 | 5.37 | 1.08 | 0.45% | 4.06 |
| Example 7D | 4 | 7.19 | 1.47 | 0.54% | 5.69 |

As can be seen in Table 10 and in FIG. 5, the DEG stayed in the filtrate/wash, along with DMI. This data shows that the processes described herein produced little DEG, a degradation product from EG.

### Example 14: Converting DMT into a TMCD-Containing Polyester

A 1000-mL RB flask, equipped with a heating mantle, a distillation column, a distillation head with thermometer, an air condenser insulated with heat tape, and a receiver flask, was charged with 340 g DMT from the above examples. DMT distillation was carried out at 45 torr vacuum and 186° C. take-off temperature. DMT product was collected as white needle (306.5 g, 99.9% GC purity).

The TMCD-Containing Polyester was prepared in quadruplicate. A typical procedure is described. A 500-mL RB flask, equipped with flask, equipped with metal stirrer, glass polymer head, an air condenser, a receiver flask, a N₂ inlet and vacuum was charged with 59.13 g TMCD methanolic solution (38 wt % concentration). The solution was carefully immersed into a metal bath and MeOH was slowly boiled off. The resulting TMCD was removed from the metal bath and cooled to ambient temperature. DMT (67.98 g) and CHDM (32.83 g) were added to the flask. FASCAT 4102 catalyst (125 ppm) was added last. The resulting mixture was immersed in a 220° C. metal bath and stirred for 15 minutes. The reaction was gradually heated to 245° C. over 50 minutes. Vacuum was reduced to 250 torr over 3 minutes. Then temperature was further increased to 277° C. over 26 minutes and vacuum was slowly pulled to 1.5 torr. The mixture was held at 277° C. for 37 minutes while gradually reducing the stir rate. After the reaction was complete, polymer was recovered as pale yellow transparent solid.

All 4 polymer products were grinded and tested for inherent viscosity and color. Then they were combined and dried. The resulting mixture was molded into color plaque (1.25″×1.25″×0.125″) using mini-jector at 285° C. The color plaque was analyzed for haze and color measurement using HunterLab UltraScan Pro Spectrophotometer. The color determinations are averages of values measured on the polymer plaques. They are determined by the L*a*b* color system of the CIE (International Commission on Illumination) (translated), wherein L* represents the lightness coordinate, a* represents the red/green coordinate, and b* represents the yellow/blue coordinate. Color values were measured following ASTM D 6290-98 and ASTM E308-99 using measurements from the HunterLab UltraScan Pro Spectrophotometer. Table 11 shows that rDMT (recycled DMT) delivered TMCD-containing polyester with slightly higher IV and comparable haze and color data. Example References 14A-14D are the four preparations of TMCD-containing polyester and associated data using the recovered DMT from the above examples. Example References 14E-14H are control TMCD-containing polyesters also prepared in quadruplicate using virgin DMT in a method similar to that described above for Example References 14A-14D.

TABLE 11
Data summary of Example 14 with respect to inherent viscosity, color, and haze

| | Pellet | | | | Plaque | | | |
|---|---|---|---|---|---|---|---|---|
| Example | IV | | | | Haze | | | |
| Reference | (dL/g) | L* | a* | b* | (%) | L* | a* | b* |
| Example 14A | 0.636 | 83.38 | −1.03 | 2.39 | 2.52 | 96.05 | −0.15 | 1.19 |
| Example 14B | 0.643 | 83.48 | −0.92 | 2.31 | | | | |
| Example 14C | 0.645 | 81.4 | −0.97 | 2.34 | | | | |
| Example 14D | 0.652 | 83.65 | −1.07 | 2.76 | | | | |
| Example 14E | 0.631 | 79.21 | −0.55 | 1.34 | 1.50 | 96.19 | −0.09 | 1.04 |
| Example 14F | 0.611 | 79.42 | −0.62 | 1.62 | | | | |
| Example 14G | 0.629 | 78.83 | −0.59 | 1.46 | | | | |
| Example 14H | 0.629 | 79.45 | −0.71 | 2.04 | | | | |

The present disclosure can also be described in accordance with the following numbered clauses.
Clause 1. A process for recovering one or more dialkyl terephthalates from a polyester composition, comprising: obtaining a polyester composition comprising one or more polyesters; exposing the polyester composition to one or more glycols under depolymerization conditions to provide a first mixture, the first mixture comprising a first liquid component and a first solid component, the first liquid component comprising one or more depolymerization products, wherein a weight ratio of the amount of the one or more glycols relative to the amount of the polyester composition is from 4:1 to 1:9; separating at least a portion of the first liquid component from the first solid component, wherein the separating occurs at a temperature of from 50° C. to 150° C.; and exposing the at least a portion of the first liquid component to an alcohol composition under conditions including a temperature of from 23° C. to 90° C. to provide a second mixture, the second mixture comprising a second liquid component and a second solid component, the second solid component comprising one or more dialkyl terephthalates.
Clause 2. The process of clause 1, wherein, after the separating at least a portion of the first liquid component from the first solid component, the at least a portion of the first liquid component is directly utilized in the step of the exposing the at least a portion of the first liquid component to an alcohol composition.
Clause 3. The process of clauses 1-2, wherein, after the separating at least a portion of the first liquid component from the first solid component, the at least a portion of the first liquid component is not subjected to a distillation process or further separation process prior to being utilized in the step of the exposing the at least a portion of the first liquid component to an alcohol composition.
Clause 4. The process of clauses 1-3, wherein the polyester composition comprises polyethylene terephthalate (PET), 1,4-cyclohexanedimethanol (CHDM)-modified PET, isophthalic acid (IPA)-modified PET, diethylene glycol (DEG)-modified PET, neopentyl glycol (NPG)-modified PET, propane diol (PDO)-modified PET, butanediol (BDO)-modified PET, heaxanediol (HDO)-modified PET, 2-methyl-2,4-pentanediol (MP diol)-modified PET, isosorbide-modified PET, poly(tetramethylene ether) glycol (PTMG)-modified PET, poly(ethylene glycol) (PEG)-modified PET, polycyclohexylenedimethylene terephthalate (PCT), cyclohexanedimethanol (CHDM)-containing copolyester, isosorbide-containing copolyester, or a combination thereof.
Clause 5. The process of clauses 1-4, wherein the polyester composition contains 0 mole % to 100 mole % CHDM, 0 mole % to 100 mole % DEG, 0 mole % to 100 mole % NPG, 0 mole % to 100 mole % PDO, 0 mole % to 100 mole % BDO, 0 mole % to 100 mole % HDO, 0 mole % to 100 mole % MP diol, 0 mole % to 100 mole % isosorbide, 0 mole % to 100 mole % PTMG, 0 mole % to 100 mole % PEG, and 0 mole % to 30 mole % isophthalic acid, wherein the sum of diol equivalents in the one or more polyesters is about 100 mole %, and wherein the sum of diacid equivalents in the one or more polyesters is about 100 mole %.
Clause 6. The process of clauses 1-5, wherein the polyester composition has an inherent viscosity of from about 0.1 dL/g to about 1.2 dL/g, as determined according to ASTM D2857-70.
Clause 7. The process of clauses 1-6, wherein the one or more polyesters present in the polyester composition are recycled polyesters.
Clause 8. The process of clauses 1-7, wherein the polyester composition comprises one or more foreign materials, the one or more foreign materials comprise at least one member selected from the group consisting of polyesters other than polyethylene terephthalate, polyvinyl chloride (PVC), polyvinyl acetal, polyvinyl butyral (PVB), polyvinyl alcohol (PVOH), ethylene vinyl alcohol (EVOH), cotton, polyolefins, polyethylene, polypropylene, polystyrene, polycarbonate, Spandex, natural fibers, cellulose ester, polyacrylates, polymethacrylate, polyamides, nylon, poly(lactic acid), polydimethylsiloxane, polysilane, calcium carbonate, titanium dioxide, inorganic fillers, dyes, pigments, color toners, colorants, plasticizers, adhesives, flame retardants, metals, aluminum, and iron.
Clause 9. The process of clause 8, wherein the one or more foreign materials are present in the polyester composition in an amount of from 0.01 wt. % to 50 wt. %, relative to the weight of the one or more polyesters in the polyester composition.
Clause 10. The process of clauses 1-9, wherein the one or more dialkyl terephthalates in the second solid component comprise dimethyl terephthalate (DMT), and wherein the DMT is at least 90% pure.
Clause 11. The process of clauses 1-10, wherein the second solid component further comprises: dimethyl isophthalate (DMI) in an amount of 1000 ppm or less, or 500 ppm or less; bisphenol A (BPA) in an amount of 1000 ppm or less, or 500 ppm or less; or both.
Clause 12. The process of clauses 1-11, wherein the depolymerization conditions include a temperature of from 150° C. to 260° C. and an absolute pressure of from 1 atmosphere (atm) to 15 atm in an agitated reactor for 0.5 h to 10 h.
Clause 13. The process of clauses 1-12, wherein the exposing the polyester composition to one or more glycols occurs in the presence of one or more catalysts, wherein the one or more catalysts are present in an amount of from 0.1 wt. % to 10 wt. %, relative to the weight of the polyester composition.
Clause 14. The process of clause 13, wherein the one or more catalysts comprise a member selected from the group consisting of Li₂CO₃, K₂CO₃, CaCO₃, Na₂CO₃, Cs₂CO₃, ZrCO3, LiOH, NaOH, KOH, tetrabutylammonium hydroxide (TBAH), sodium methoxide (NaOMe), lithium methoxide (LiOMe), magnesium methoxide (Mg(OMe)₂, potassium t-butoxide, ethylene glycol monosodium salt, ethylene glycol disodium salt, tetra isopropyl titanate (TIPT), butyltin tris-2-ethylhexanoate (FASCAT 4102), 1,8-Diazabicyclo[5.4.0]undec-7-ene (DBU), zinc acetylacetonate hydrate (Zn(acac)₂), zinc acetate (Zn(OAc)₂), manganese (II) acetate (Mn(OAc)₂), hydrotalcite, zeolite, and lithium chloride.
Clause 15. The process of clause 13, wherein the one or more catalysts comprise a member selected from the group consisting of LiOH, NaOH, KOH, tetra isopropyl titanate (TIPT), butyltin tris-2-ethylhexanoate (FASCAT 4102), ZrCO₃, 1,8-Diazabicyclo[5.4.0]undec-7-ene (DBU), sodium methoxide (NaOMe), lithium methoxide (LiOMe), zinc acetylacetonate hydrate (Zn(acac)₂), Cs₂CO₃, ethylene glycol sodium salt, manganese (II) acetate (Mn(OAc)₂), hydrotalcite, and zeolite.
Clause 16. The process of clause 13, wherein the one or more catalysts comprise a member selected from the group consisting of LiOH, NaOH, KOH, sodium methoxide (NaOMe), Cs₂CO₃, ethylene glycol sodium salt, lithium methoxide (LiOMe), hydrotalcite, and zeolite.
Clause 17. The process of clauses 1-16, wherein the one or more glycols comprises ethylene glycol (EG), diethylene glycol (DEG), triethylene glycol (TEG), 1,4-cyclohexanedimethanol (CHDM), poly(ethylene glycol) (PEG), neopentyl glycol (NPG), propane diol (PDO), butanediol (BDO), 2-methyl-2,4-pentanediol (MP diol), poly(tetramethylene ether)glycol (PTMG), or a combination thereof.
Clause 18. The process of clauses 1-17, wherein the one or more glycols comprises 0-100% ethylene glycol (EG), 0-100% diethylene glycol (DEG), 0-100% triethylene glycol (TEG), 0-100% poly(ethylene glycol) (PEG), 0-100% neopentyl glycol (NPG), 0-100% propane diol (PDO), 0-100% butanediol (BDO), 0-100% 2-methyl-2,4-pentanediol (MP diol), 0-100% poly(tetramethylene ether)glycol (PTMG), and 0-50% CHDM.
Clause 19. The process of clause 1-18, wherein the separating comprises filtration, centrifugation, settling, sedimentation, or a combination thereof.
Clause 20. The process of clauses 1-19, wherein the alcohol composition comprises methanol.
Clause 21. The process of clauses 1-20, wherein, in the step of the exposing at least a portion of the first liquid component to an alcohol composition, a weight ratio of the amount of the alcohol composition relative to the amount of the polyester composition is from 2:1 to 10:1.
Clause 22. The process of clauses 1-21, wherein, the exposing at least a portion of the first liquid component to an alcohol composition occurs in the presence of one or more alcoholysis catalysts.
Clause 23. The process of clause 22, wherein, the one or more alcoholysis catalysts is present in an amount of from 0.1 wt. % to 20 wt. %, relative to the weight of the polyester composition.
Clause 24. The process of clauses 22-23, wherein the one or more alcoholysis catalysts comprise K₂CO₃, Na₂CO₃, Li₂CO₃, Cs₂CO₃; KOH, LiOH, NaOH; NaOMe, Mg(OMe)₂, KOMe, KOt-Bu, ethylene glycol monosodium salt, ethylene glycol disodium salt, or a combination thereof.
Clause 25. The process of clauses 22-23, wherein the one or more alcoholysis catalysts comprise KOH, NaOH, NaOMe or a combination thereof, and wherein the one or more alcoholysis catalysts are in a solid form, a solution form in water, methanol, or ethylene glycol, or a combination of thereof.
Clause 26. The process of clauses 1-25, further comprising: separating the second solid component from the second liquid component.
Clause 27. The process of clauses 1-26, wherein the second liquid component comprises at least a portion of the one or more glycols, at least a portion of the alcohol composition, diethylene glycol (DEG), 1,4-cyclohexanedimethanol (CHDM), or a combination thereof.
Clause 28. The process of clause 26, further comprising: separating, from the second liquid component, one or more of the at least a portion of the one or more glycols, at least a portion of the alcohol composition, diethylene glycol, or 1,4-cyclohexanedimethanol (CHDM).
Clause 29. The process of clauses 1-28, further comprising: depolymerizing at least a portion of one or more second polyesters in a second polyester composition by exposing the second polyester composition to at least a portion of the second liquid component.
Clause 30. The process of clauses 1-29, wherein the process is conducted as a batch process, a semi-continuous process, or a continuous process.
Clause 31. The process of clauses 1-30, wherein, in the obtaining the polyester composition, the polyester composition is in solid form, molten form, liquid form, or solution form.
Clause 32. The process of clauses 8-9, wherein, at least a portion of the one or more foreign materials is present in the first solid component of the first mixture.
Clause 33. The process of clauses 1-32, wherein the one or more depolymerization products comprise monomers, oligomers, or a combination thereof.
Clause 34. The process of clause 33, wherein the oligomers exhibit a degree of polymerization of from 2 to 10.
Clause 35. The process of clauses 1-34, wherein the one or more glycols comprise ethylene glycol (EG), and wherein, in the step of the exposing the polyester composition to one or more glycols, less than 5 wt. % of dietheylene glycol (DEG) is produced.
Clause 36. A process for recovering one or more dialkyl terephthalates from a polyester composition, comprising: obtaining a polyester composition comprising one or more polyesters; exposing the polyester composition to one or more glycols under depolymerization conditions to provide a first mixture, the first mixture comprising a first liquid component and a first solid component, the first liquid component comprising one or more depolymerization products, wherein the one or more depolymerization products comprise monomers, oligomers, or a combination thereof; separating at least a portion of the first liquid component from the first solid component, wherein the separating occurs at a temperature of 150° C. or less; and exposing the at least a portion of the first liquid component to an alcohol composition under conditions including a temperature of 90° C. or less to provide a second mixture, the second mixture comprising a second liquid component and a second solid component, the second solid component comprising one or more dialkyl terephthalates.
Clause 37. The process of clause 36, wherein, after the separating at least a portion of the first liquid component from the first solid component, the at least a portion of the first liquid component is directly utilized in the step of the exposing the at least a portion of the first liquid component to an alcohol composition.
Clause 38. The process of clauses 36-37, wherein, after the separating at least a portion of the first liquid component from the first solid component, the at least a portion of the first liquid component is not subjected to a distillation process or further separation process prior to being utilized in the step of the exposing the at least a portion of the first liquid component to an alcohol composition.
Clause 39. The process of clauses 36-38, wherein the polyester composition comprises polyethylene terephthalate (PET), 1,4-cyclohexanedimethanol (CHDM)-modified PET, isophthalic acid (IPA)-modified PET, diethylene glycol (DEG)-modified PET, neopentyl glycol (NPG)-modified PET, propane diol (PDO)-modified PET, butanediol (BDO)-modified PET, heaxanediol (HDO)-modified PET, 2-methyl-2,4-pentanediol (MP diol)-modified PET, isosorbide-modified PET, poly(tetramethylene ether) glycol (PTMG)-modified PET, poly(ethylene glycol) (PEG)-modified PET, polycyclohexylenedimethylene terephthalate (PCT), cyclohexanedimethanol (CHDM)-containing copolyester, isosorbide-containing copolyester, or a combination thereof.
Clause 40. The process of clauses 36-39, wherein the polyester composition contains 0 mole % to 100 mole % CHDM, 0 mole % to 100 mole % DEG, 0 mole % to 100 mole % NPG, 0 mole % to 100 mole % PDO, 0 mole % to 100 mole % BDO, 0 mole % to 100 mole % HDO, 0 mole % to 100 mole % MP diol, 0 mole % to 100 mole % isosorbide, 0 mole % to 100 mole % PTMG, 0 mole % to 100 mole % PEG, and 0 mole % to 30 mole % isophthalic acid, wherein the sum of diol equivalents in the one or more polyesters is about 100 mole %, and wherein the sum of diacid equivalents in the one or more polyesters is about 100 mole %.
Clause 41. The process of clauses 36-40, wherein the polyester composition has an inherent viscosity of from about 0.1 dL/g to about 1.2 dL/g, as determined according to ASTM D2857-70.
Clause 42. The process of clauses 36-41, wherein the one or more polyesters present in the polyester composition are recycled polyesters.
Clause 43. The process of clauses 36-42, wherein the polyester composition comprises one or more foreign materials, the one or more foreign materials comprise at least one member selected from the group consisting of polyesters other than polyethylene terephthalate, polyvinyl chloride (PVC), polyvinyl acetal, polyvinyl butyral (PVB), polyvinyl alcohol (PVOH), ethylene vinyl alcohol (EVOH), cotton, polyolefins, polyethylene, polypropylene, polystyrene, polycarbonate, Spandex, natural fibers, cellulose ester, polyacrylates, polymethacrylate, polyamides, nylon, poly(lactic acid), polydimethylsiloxane, polysilane, calcium carbonate, titanium dioxide, inorganic fillers, dyes, pigments, color toners, colorants, plasticizers, adhesives, flame retardants, metals, aluminum, and iron.
Clause 44. The process of clause 43, wherein the one or more foreign materials are present in the polyester composition in an amount of from 0.01 wt. % to 50 wt. %, relative to the weight of the one or more polyesters in the polyester composition.
Clause 45. The process of clauses 36-44, wherein the one or more dialkyl terephthalates in the second solid component comprise dimethyl terephthalate (DMT), and wherein the DMT is at least 90% pure.
Clause 46. The process of clauses 36-45, wherein the second solid component further comprises: dimethyl isophthalate (DMI) in an amount of 1000 ppm or less, or 500 ppm or less; bisphenol A (BPA) in an amount of 1000 ppm or less, or 500 ppm or less; or both.
Clause 47. The process of clauses 36-46, wherein the depolymerization conditions include a temperature of from 150° C. to 260° C. and an absolute pressure of from 1 atmosphere (atm) to 15 atm in an agitated reactor for 0.5 h to 10 h.
Clause 48. The process of clauses 36-47, wherein the exposing the polyester composition to one or more glycols occurs in the presence of one or more catalysts, wherein the one or more catalysts are present in an amount of from 0.1 wt. % to 10 wt. %, relative to the weight of the polyester composition.
Clause 49. The process of clause 48, wherein the one or more catalysts comprise a member selected from the group consisting of Li₂CO₃, K₂CO₃, CaCO₃, Na₂CO₃, Cs₂CO₃, ZrCO3, LiOH, NaOH, KOH, tetrabutylammonium hydroxide (TBAH), sodium methoxide (NaOMe), lithium methoxide (LiOMe), magnesium methoxide (Mg(OMe)₂, potassium t-butoxide, ethylene glycol monosodium salt, ethylene glycol disodium salt, tetra isopropyl titanate (TIPT), butyltin tris-2-ethylhexanoate (FASCAT 4102), 1,8-Diazabicyclo[5.4.0]undec-7-ene (DBU), zinc acetylacetonate hydrate (Zn(acac)₂), zinc acetate (Zn(OAc)₂), manganese (II) acetate (Mn(OAc)₂), hydrotalcite, zeolite, and lithium chloride.
Clause 50. The process of clause 48, wherein the one or more catalysts comprise a member selected from the group consisting of LiOH, NaOH, KOH, tetra isopropyl titanate (TIPT), butyltin tris-2-ethylhexanoate (FASCAT 4102), ZrCO₃, 1,8-Diazabicyclo[5.4.0]undec-7-ene (DBU), sodium methoxide (NaOMe), lithium methoxide (LiOMe), zinc acetylacetonate hydrate (Zn(acac)₂), Cs₂CO₃, ethylene glycol sodium salt, manganese (II) acetate (Mn(OAc)₂), hydrotalcite, and zeolite.
Clause 51. The process of clause 48, wherein the one or more catalysts comprise a member selected from the group consisting of LiOH, NaOH, KOH, sodium methoxide (NaOMe), Cs₂CO₃, ethylene glycol sodium salt, lithium methoxide (LiOMe), hydrotalcite, and zeolite.
Clause 52. The process of clauses 36-51, wherein the one or more glycols comprises ethylene glycol (EG), diethylene glycol (DEG), triethylene glycol (TEG), 1,4-cyclohexanedimethanol (CHDM), poly(ethylene glycol) (PEG), neopentyl glycol (NPG), propane diol (PDO), butanediol (BDO), 2-methyl-2,4-pentanediol (MP diol), poly(tetramethylene ether)glycol (PTMG), or a combination thereof.
Clause 53. The process of clauses 36-52, wherein the one or more glycols comprises 0-100% ethylene glycol (EG), 0-100% diethylene glycol (DEG), 0-100% triethylene glycol (TEG), 0-100% poly(ethylene glycol) (PEG), 0-100% neopentyl glycol (NPG), 0-100% propane diol (PDO), 0-100% butanediol (BDO), 0-100% 2-methyl-2,4-pentanediol (MP diol), 0-100% poly(tetramethylene ether)glycol (PTMG), and 0-50% CHDM.
Clause 54. The process of clause 36-53, wherein a weight ratio of the amount of the one or more glycols relative to the amount of the polyester composition is from 4:1 to 1:9.
Clause 55. The process of clauses 36-54, wherein the alcohol composition comprises methanol.
Clause 56. The process of clauses 36-55, wherein, in the step of the exposing at least a portion of the first liquid component to an alcohol composition, a weight ratio of the amount of the alcohol composition relative to the amount of the polyester composition is from 2:1 to 10:1.
Clause 57. The process of clauses 36-56, wherein, the exposing at least a portion of the first liquid component to an alcohol composition occurs in the presence of one or more alcoholysis catalysts.
Clause 58. The process of clause 57, wherein, the one or more alcoholysis catalysts is present in an amount of from 0.1 wt. % to 20 wt. %, relative to the weight of the polyester composition.
Clause 59. The process of clauses 57-58, wherein the one or more alcoholysis catalysts comprise K₂CO₃, Na₂CO₃, Li₂CO₃, Cs₂CO₃; KOH, LiOH, NaOH; NaOMe, Mg(OMe)₂, KOMe, KOt-Bu, ethylene glycol monosodium salt, ethylene glycol disodium salt, or a combination thereof.
Clause 60. The process of clauses 57-58, wherein the one or more alcoholysis catalysts comprise KOH, NaOH, NaOMe or a combination thereof, and wherein the one or more alcoholysis catalysts are in a solid form, a solution form in water, methanol, or ethylene glycol, or a combination of thereof.
Clause 61. The process of clauses 36-60, further comprising: separating the second solid component from the second liquid component.
Clause 62. The process of clauses 36-61, wherein the second liquid component comprises at least a portion of the one or more glycols, at least a portion of the alcohol composition, diethylene glycol, 1,4-cyclohexanedimethanol (CHDM), or a combination thereof.
Clause 63. The process of clause 62, further comprising: separating, from the second liquid component, one or more of the at least a portion of the one or more glycols, at least a portion of the alcohol composition, diethylene glycol, or 1,4-cyclohexanedimethanol (CHDM).
Clause 64. The process of clauses 36-63, further comprising:
depolymerizing at least a portion of one or more second polyesters in a second polyester composition by exposing the second polyester composition to at least a portion of the second liquid component.
Clause 65. The process of clauses 36-64, wherein the process is conducted as a batch process, a semi-continuous process, or a continuous process.
Clause 66. The process of clauses 36-65, wherein, in the obtaining the polyester composition, the polyester composition is in solid form, liquid form, or solution form.
Clause 67. The process of clauses 43-44, wherein, at least a portion of the one or more foreign materials is present in the first solid component of the first mixture.
Clause 68. The process of clauses 36-67, wherein the oligomers exhibit a degree of polymerization of from 2 to 10.
Clause 69. The process of clauses 36-68, wherein the one or more glycols comprise ethylene glycol (EG), and wherein, in the step of the exposing the polyester composition to one or more glycols, less than 5 wt. % of dietheylene glycol (DEG) is produced.
Clause 70. A process for recovering one or more dialkyl terephthalates from a polyester composition, comprising: (a) exposing a first polyester composition to one or more glycols under depolymerization conditions to provide a first mixture, the first mixture comprising a first liquid component and a first solid component, the first liquid component comprising one or more depolymerization products; (b) exposing at least a portion of the first liquid component to a first alcohol composition under alcoholysis conditions comprising a temperature of 90° C. or less to provide a second mixture, the second mixture comprising a second liquid component and a second solid component, the second solid component comprising one or more dialkyl terephthalates; (c) exposing the second liquid component to distillation conditions to provide one or more recycle glycols, the one or more recycle glycols comprising at least a portion of the one or more glycols; and (d) exposing a second polyester composition to at least a portion of the one or more recycle glycols under depolymerization conditions to provide a third mixture, the third mixture comprising a third liquid component and a third solid component, the third liquid component comprising one or more depolymerization products.
Clause 71. The process of clause 70, further comprising: (e) exposing at least a portion of the third liquid component to a second alcohol composition under alcoholysis conditions to provide a fourth mixture, the fourth mixture comprising a fourth liquid component and a fourth solid component, the fourth solid component comprising one or more dialkyl terephthalates.
Clause 72. The process of clause 71, wherein the one or more dialkyl terephthalates in the second solid component comprises dimethyl terephthalate (DMT) that is at least 90% pure, and wherein the one or more dialkyl terephthalates in the fourth solid component comprises DMT that is at least 90% pure.
Clause 73. The process of clauses 70-72, wherein the distillation conditions comprise a temperature of from 70° C. to 220° C.
Clause 74. The process of clauses 70-73, wherein the distillation conditions comprise a pressure of from 1 Torr to 800 Torr.
Clause 75. The process of clauses 70-74, wherein the one or more recycle glycols obtained from the step (c) are present in a distillation pot residue.
Clause 76. The process of clauses 70-75, further comprising, prior to the step (b), separating the at least a portion of the first liquid component from the first solid component, wherein after the separating, the at least a portion of the first liquid component is directly utilized in the step (b) of the exposing the at least a portion of the first liquid component to the first alcohol composition.
Clause 77. The process of clauses 70-75, further comprising, prior to the step (b), separating the at least a portion of the first liquid component from the first solid component, wherein after the separating, the at least a portion of the first liquid component is not subjected to a distillation process or further separation process prior to being utilized in the step (b) of the exposing the at least a portion of the first liquid component to the first alcohol composition.
Clause 78. The process of clauses 70-77, wherein the first polyester composition and the second polyester composition comprise polyethylene terephthalate (PET), 1,4-cyclohexanedimethanol (CHDM)-modified PET, isophthalic acid (IPA)-modified PET, diethylene glycol (DEG)-modified PET, neopentyl glycol (NPG)-modified PET, propane diol (PDO)-modified PET, butanediol (BDO)-modified PET, heaxanediol (HDO)-modified PET, 2-methyl-2,4-pentanediol (MP diol)-modified PET, isosorbide-modified PET, poly(tetramethylene ether) glycol (PTMG)-modified PET, poly(ethylene glycol) (PEG)-modified PET, polycyclohexylenedimethylene terephthalate (PCT), cyclohexanedimethanol (CHDM)-containing copolyester, isosorbide-containing copolyester, or a combination thereof.
Clause 79. The process of clauses 70-78, wherein the first polyester composition and the second polyester composition each contain 0 mole % to 100 mole % CHDM, 0 mole % to 100 mole % DEG, 0 mole % to 100 mole % NPG, 0 mole % to 100 mole % PDO, 0 mole % to 100 mole % BDO, 0 mole % to 100 mole % HDO, 0 mole % to 100 mole % MP diol, 0 mole % to 100 mole % isosorbide, 0 mole % to 100 mole % PTMG, 0 mole % to 100 mole % PEG, and 0 mole % to 30 mole % isophthalic acid, wherein the sum of diol equivalents in each of the first polyester composition and the second polyester composition is about 100 mole %, and wherein the sum of diacid equivalents in each of the first polyester composition and the second polyester composition is about 100 mole %.
Clause 80. The process of clauses 70-79, wherein the first polyester composition and the second polyester composition have an inherent viscosity of from about 0.1 dL/g to about 1.2 dL/g, as determined according to ASTM D2857-70.
Clause 81. The process of clauses 70-80, wherein one or more polyesters present in the first polyester composition, the second polyester composition, or both, are recycled polyesters.
Clause 82. The process of clauses 70-81, wherein the first polyester composition, the second polyester composition, or both, comprise one or more foreign materials, the one or more foreign materials comprise at least one member selected from the group consisting of polyesters other than polyethylene terephthalate, polyvinyl chloride (PVC), polyvinyl acetal, polyvinyl butyral (PVB), polyvinyl alcohol (PVOH), ethylene vinyl alcohol (EVOH), cotton, polyolefins, polyethylene, polypropylene, polystyrene, polycarbonate, Spandex, natural fibers, cellulose ester, polyacrylates, polymethacrylate, polyamides, nylon, poly(lactic acid), polydimethylsiloxane, polysilane, calcium carbonate, titanium dioxide, inorganic fillers, dyes, pigments, color toners, colorants, plasticizers, adhesives, flame retardants, metals, aluminum, and iron.
Clause 83. The process of clause 82, wherein the one or more foreign materials are present in the first polyester composition, the second polyester composition, or both, in an amount of from 0.01 wt. % to 10 wt. %, relative to the weight of polyester in the polyester composition.
Clause 84. The process of clauses 70-83, wherein the one or more dialkyl terephthalates in the second solid component comprise dimethyl terephthalate (DMT), and wherein the DMT is at least 90% pure.
Clause 85. The process of clauses 70-84, wherein the second solid component further comprises: dimethyl isophthalate (DMI) in an amount of 1000 ppm or less, or 500 ppm or less; bisphenol A (BPA) in an amount of 1000 ppm or less, or 500 ppm or less; or both.
Clause 86. The process of clauses 70-85, wherein the depolymerization conditions include a temperature of from 150° C. to 260° C. and an absolute pressure of from 1 atmosphere (atm) to 15 atm in an agitated reactor for 0.5 h to 10 h.
Clause 87. The process of clauses 70-86, wherein the exposing the first polyester composition to one or more glycols occurs in the presence of one or more catalysts, wherein the one or more catalysts are present in an amount of from 0.1 wt. % to 10 wt. %, relative to the weight of the first polyester composition.
Clause 88. The process of clause 87, wherein the one or more catalysts comprise a member selected from the group consisting of Li₂CO₃, K₂CO₃, CaCO₃, Na₂CO₃, Cs₂CO₃, ZrCO3, LiOH, NaOH, KOH, tetrabutylammonium hydroxide (TBAH), sodium methoxide (NaOMe), lithium methoxide (LiOMe), magnesium methoxide (Mg(OMe)₂, potassium t-butoxide, ethylene glycol monosodium salt, ethylene glycol disodium salt, tetra isopropyl titanate (TIPT), butyltin tris-2-ethylhexanoate (FASCAT 4102), 1,8-Diazabicyclo[5.4.0]undec-7-ene (DBU), zinc acetylacetonate hydrate (Zn(acac)₂), zinc acetate (Zn(OAc)₂), manganese (II) acetate (Mn(OAc)₂), hydrotalcite, zeolite, and lithium chloride.
Clause 89. The process of clause 87, wherein the one or more catalysts comprise a member selected from the group consisting of LiOH, NaOH, KOH, tetra isopropyl titanate (TIPT), butyltin tris-2-ethylhexanoate (FASCAT 4102), ZrCO₃, 1,8-Diazabicyclo[5.4.0]undec-7-ene (DBU), sodium methoxide (NaOMe), lithium methoxide (LiOMe), Cs₂CO₃, ethylene glycol sodium salt, zinc acetylacetonate hydrate (Zn(acac)₂), manganese (II) acetate (Mn(OAc)₂), hydrotalcite, and zeolite.
Clause 90. The process of clause 87, wherein the one or more catalysts comprise a member selected from the group consisting of LiOH, NaOH, KOH, sodium methoxide (NaOMe), lithium methoxide (LiOMe), Cs₂CO₃, ethylene glycol sodium salt, hydrotalcite, and zeolite.
Clause 91. The process of clauses 70-90, wherein the one or more glycols comprises ethylene glycol (EG), diethylene glycol (DEG), triethylene glycol (TEG), 1,4-cyclohexanedimethanol (CHDM), poly(ethylene glycol) (PEG), neopentyl glycol (NPG), propane diol (PDO), butanediol (BDO), 2-methyl-2,4-pentanediol (MP diol), poly(tetramethylene ether)glycol (PTMG), or a combination thereof.
Clause 92. The process of clauses 70-91, wherein the one or more glycols comprises 0-100% ethylene glycol (EG), 0-100% diethylene glycol (DEG), 0-100% triethylene glycol (TEG), 0-100% poly(ethylene glycol) (PEG), 0-100% neopentyl glycol (NPG), 0-100% propane diol (PDO), 0-100% butanediol (BDO), 0-100% 2-methyl-2,4-pentanediol (MP diol), 0-100% poly(tetramethylene ether)glycol (PTMG), and 0-50% CHDM.
Clause 93. The process of clause 70-92, wherein a weight ratio of the amount of the one or more glycols relative to the amount of the first polyester composition is from 3:1 to 1:9.
Clause 94. The process of clauses 70-93, wherein the first alcohol composition comprises methanol.
Clause 95. The process of clauses 70-94, wherein, in the step (b) of exposing at least a portion of the first liquid component to the first alcohol composition, a weight ratio of the amount of the first alcohol composition relative to the amount of the first polyester composition is from 2:1 to 10:1.
Clause 96. The process of clauses 70-95, wherein, in the step (b) of exposing at least a portion of the first liquid component to the first alcohol composition occurs in the presence of one or more alcoholysis catalysts.
Clause 97. The process of clause 96, wherein, the one or more alcoholysis catalysts is present in an amount of from 0.1 wt. % to 20 wt. %, relative to the weight of the first polyester composition.
Clause 98. The process of clauses 96-97, wherein the one or more alcoholysis catalysts comprise K₂CO₃, Na₂CO₃, Li₂CO₃, Cs₂CO₃; KOH, LiOH, NaOH; NaOMe, Mg(OMe)₂, KOMe, KOt-Bu, ethylene glycol monosodium salt, ethylene glycol disodium salt, or a combination thereof.
Clause 99. The process of clauses 96-97, wherein the one or more alcoholysis catalysts comprise KOH, NaOH, NaOMe or a combination thereof, and wherein the one or more alcoholysis catalysts are in a solid form, a solution form in water, methanol, or ethylene glycol, or a combination of thereof.
Clause 100. The process of clauses 70-99, further comprising, prior to the step (c), separating the second solid component from the second liquid component.
Clause 101. The process of clauses 70-100, wherein the second liquid component comprises at least a portion of the one or more glycols, at least a portion of the alcohol composition, diethylene glycol, 1,4-cyclohexanedimethanol (CHDM), or a combination thereof.
Clause 102. The process of clause 70, further comprising, prior to the step (b), separating the at least a portion of the first liquid component from the first solid component, and wherein the separating occurs at a temperature of from 50° C. to 150° C.
Clause 103. The process of clauses 70-102, wherein the process is conducted as a batch process, a semi-continuous process, or a continuous process.
Clause 104. The process of clauses 82-83, wherein, at least a portion of the one or more foreign materials is present in the first solid component of the first mixture.
Clause 105. The process of clauses 70-104, wherein the one or more depolymerization products comprise monomers, oligomers, or a combination thereof.
Clause 106. The process of clause 105, wherein the oligomers exhibit a degree of polymerization of from 2 to 10.
Clause 107. The process of clauses 70-106, wherein the one or more glycols comprise ethylene glycol (EG), and wherein, in the step (a) of the exposing the first polyester composition to one or more glycols, less than 5 wt. % of dietheylene glycol (DEG) is produced.
Clause 108. The process of clause 87, wherein the one or more catalysts are added during at least a portion of the step (a), and wherein no additional catalyst is added during the step (d).
Clause 109. The process of clauses 70-108, further comprising, prior to the step (b), separating the at least a portion of the first liquid component from the first solid component.

This disclosure has been described in detail with particular reference to specific aspects thereof, but it will be understood that variations and modifications can be made within the spirit and scope of this disclosure.

1. A process for recovering one or more dialkyl terephthalates from a polyester composition, comprising: (a) exposing a first polyester composition to one or more glycols under depolymerization conditions to provide a first mixture, the first mixture comprising a first liquid component and a first solid component, the first liquid component comprising one or more depolymerization products; (b) exposing at least a portion of the first liquid component to a first alcohol composition under alcoholysis conditions comprising a temperature of 90° C. or less to provide a second mixture, the second mixture comprising a second liquid component and a second solid component, the second solid component comprising one or more dialkyl terephthalates; (c) exposing the second liquid component to distillation conditions to provide one or more recycle glycols, the one or more recycle glycols comprising at least a portion of the one or more glycols; and (d) exposing a second polyester composition to at least a portion of the one or more recycle glycols under depolymerization conditions to provide a third mixture, the third mixture comprising a third liquid component and a third solid component, the third liquid component comprising one or more depolymerization products.

2. The process of claim 1, further comprising: (e) exposing at least a portion of the third liquid component to a second alcohol composition under alcoholysis conditions to provide a fourth mixture, the fourth mixture comprising a fourth liquid component and a fourth solid component, the fourth solid component comprising one or more dialkyl terephthalates.

3. The process of claim 2, wherein the one or more dialkyl terephthalates in the second solid component comprises dimethyl terephthalate (DMT) that is at least 90% pure, and wherein the one or more dialkyl terephthalates in the fourth solid component comprises DMT that is at least 90% pure.

4. The process of claim 1, wherein the distillation conditions comprise a temperature of from 70° C. to 220° C., wherein the distillation conditions comprise a pressure of from 1 Torr to 800 Torr, or both.

5. The process of claim 1, wherein the one or more recycle glycols obtained from the step (c) are present in a distillation pot residue.

6. The process of claim 5, further comprising, prior to the step (b), separating the at least a portion of the first liquid component from the first solid component, wherein after the separating, the at least a portion of the first liquid component is directly utilized in the step (b) of the exposing the at least a portion of the first liquid component to the first alcohol composition; or further comprising, prior to the step (b), separating the at least a portion of the first liquid component from the first solid component, wherein after the separating, the at least a portion of the first liquid component is not subjected to a distillation process or further separation process prior to being utilized in the step (b) of the exposing the at least a portion of the first liquid component to the first alcohol composition.

7. The process of claim 1, wherein the first polyester composition and the second polyester composition comprise polyethylene terephthalate (PET), 1,4-cyclohexanedimethanol (CHDM)-modified PET, isophthalic acid (IPA)-modified PET, diethylene glycol (DEG)-modified PET, neopentyl glycol (NPG)-modified PET, propane diol (PDO)-modified PET, butanediol (BDO)-modified PET, heaxanediol (HDO)-modified PET, 2-methyl-2,4-pentanediol (MP diol)-modified PET, isosorbide-modified PET, poly(tetramethylene ether) glycol (PTMG)-modified PET, poly(ethylene glycol) (PEG)-modified PET, polycyclohexylenedimethylene terephthalate (PCT), cyclohexanedimethanol (CHDM)-containing copolyester, isosorbide-containing copolyester, or a combination thereof.

8. The process of claim 1, wherein the first polyester composition and the second polyester composition each contain 0 mole % to 100 mole % CHDM, 0 mole % to 100 mole % DEG, 0 mole % to 100 mole % NPG, 0 mole % to 100 mole % PDO, 0 mole % to 100 mole % BDO, 0 mole % to 100 mole % HDO, 0 mole % to 100 mole % MP diol, 0 mole % to 100 mole % isosorbide, 0 mole % to 100 mole % PTMG, 0 mole % to 100 mole % PEG, and 0 mole % to 30 mole % isophthalic acid, wherein the sum of diol equivalents in each of the first polyester composition and the second polyester composition is about 100 mole %, and wherein the sum of diacid equivalents in each of the first polyester composition and the second polyester composition is about 100 mole %.

9. The process of claim 1, wherein the first polyester composition, the second polyester composition, or both, comprise one or more foreign materials, the one or more foreign materials comprise at least one member selected from the group consisting of polyesters other than polyethylene terephthalate, polyvinyl chloride (PVC), polyvinyl acetal, polyvinyl butyral (PVB), polyvinyl alcohol (PVOH), ethylene vinyl alcohol (EVOH), cotton, polyolefins, polyethylene, polypropylene, polystyrene, polycarbonate, Spandex, natural fibers, cellulose ester, polyacrylates, polymethacrylate, polyamides, nylon, poly(lactic acid), polydimethylsiloxane, polysilane, calcium carbonate, titanium dioxide, inorganic fillers, dyes, pigments, color toners, colorants, plasticizers, adhesives, flame retardants, metals, aluminum, and iron, and wherein, at least a portion of the one or more foreign materials is present in the first solid component of the first mixture.

10. The process of claim 9, wherein the one or more foreign materials are present in the first polyester composition, the second polyester composition, or both, in an amount of from 0.01 wt. % to 10 wt. %, relative to the weight of polyester in the polyester composition.

11. The process of claim 1, wherein the one or more dialkyl terephthalates in the second solid component comprise dimethyl terephthalate (DMT), and wherein the DMT is at least 90% pure.

12. The process of claim 1, wherein the second solid component further comprises: dimethyl isophthalate (DMI) in an amount of 1000 ppm or less, or 500 ppm or less; bisphenol A (BPA) in an amount of 1000 ppm or less, or 500 ppm or less; or both.

13. The process of claim 1, wherein the depolymerization conditions include a temperature of from 150° C. to 260° C. and an absolute pressure of from 1 atmosphere (atm) to 15 atm in an agitated reactor for 0.5 h to 10 h.

14. The process of claim 1, wherein the exposing the first polyester composition to one or more glycols occurs in the presence of one or more catalysts, wherein the one or more catalysts are present in an amount of from 0.1 wt. % to 10 wt. %, relative to the weight of the first polyester composition, and wherein the one or more catalysts are added during at least a portion of the step (a), and wherein no additional catalyst is added during the step (d).

15. The process of claim 14, wherein the one or more catalysts comprise a member selected from the group consisting of Li₂CO₃, K₂CO₃, CaCO₃, Na₂CO₃, Cs₂CO₃, ZrCO3, LiOH, NaOH, KOH, tetrabutylammonium hydroxide (TBAH), sodium methoxide (NaOMe), lithium methoxide (LiOMe), magnesium methoxide (Mg(OMe)₂, potassium t-butoxide, ethylene glycol monosodium salt, ethylene glycol disodium salt, tetra isopropyl titanate (TIPT), butyltin tris-2-ethylhexanoate (FASCAT 4102), 1,8-Diazabicyclo[5.4.0]undec-7-ene (DBU), zinc acetylacetonate hydrate (Zn(acac)₂), zinc acetate (Zn(OAc)₂), manganese (II) acetate (Mn(OAc)₂), hydrotalcite, zeolite, and lithium chloride.

16. The process of claim 1, wherein the one or more glycols comprises ethylene glycol (EG), diethylene glycol (DEG), triethylene glycol (TEG), 1,4-cyclohexanedimethanol (CHDM), poly(ethylene glycol) (PEG), neopentyl glycol (NPG), propane diol (PDO), butanediol (BDO), 2-methyl-2,4-pentanediol (MP diol), poly(tetramethylene ether)glycol (PTMG), or a combination thereof.

17. The process of claim 1, wherein a weight ratio of the amount of the one or more glycols relative to the amount of the first polyester composition is from 3:1 to 1:9.

18. The process of claim 1, wherein the first alcohol composition comprises methanol, and wherein, in the step (b) of exposing at least a portion of the first liquid component to the first alcohol composition, a weight ratio of the amount of the first alcohol composition relative to the amount of the first polyester composition is from 2:1 to 10:1.

19. The process of 1, wherein, in the step (b) of exposing at least a portion of the first liquid component to the first alcohol composition occurs in the presence of one or more alcoholysis catalysts, wherein, the one or more alcoholysis catalysts is present in an amount of from 0.1 wt. % to 20 wt. %, relative to the weight of the first polyester composition, and wherein the one or more alcoholysis catalysts comprise K₂CO₃, Na₂CO₃, Li₂CO₃, Cs₂CO₃;
KOH, LiOH, NaOH; NaOMe, Mg(OMe)₂, KOMe, KOt-Bu, ethylene glycol monosodium salt, ethylene glycol disodium salt, or a combination thereof.

20. The process of claim 1, further comprising, prior to the step (b), separating the at least a portion of the first liquid component from the first solid component, and wherein the separating occurs at a temperature of from 50° C. to 150° C.
